(19) United States
(12) Patent Application Publication
(10) Pub. No.: US 2026/0041363 A1
(43) Pub. Date: Feb. 12, 2026

(54) SYSTEM AND METHOD FOR LONGITUDINALASSESSMENT OF NERVE HEALTH

(71) Applicant: NEURALYTIX, LLC, Fruita, CO (US)

(72) Inventors: David S. Nay, Novi, MI (US); Christopher Wybo, Colorado Springs, CO (US)

(73) Assignee: NEURALYTIX, LLC, Fruita, CO (US)

(21) Appl. No.: 19/296,714

(22) Filed: Aug. 11, 2025

Related U.S. Application Data
(60) Provisional application No. 63/681,766, filed on Aug. 9, 2024.
(60) Provisional application No. 63/792,881, filed on Apr. 22, 2025.

(51) Int. Cl. A61B 5/00; A61B 5/11; A61B 6/00; A61N 1/05

(52) U.S. Cl. CPC A61B 5/4041; A61B 5/1104; A61B 5/4519; A61B 5/6804; A61B 5/7267; A61B 5/742; A61B 6/485; A61N 1/0551; A61B 2562/0219

(57) ABSTRACT

A system for longitudinal assessment of nerve health includes a stimulator to deliver electrical stimuli proximate a target nerve, a sensor to detect muscle responses evoked by the stimuli, a display, and a processor in communication with the stimulator, sensor, and display. The processor controls delivery of the stimuli; determines, from the detected muscle response, one or more nerve-function parameters for the target nerve including at least a stimulation threshold; stores the parameters with a session identifier; retrieves parameters from multiple sessions to compute a longitudinal trend; and controls the display to present the trend together with an indication of the target nerve's status relative to a target range. The system supports objective session-to-session tracking to inform clinical decisions.

### CROSS REFERENCE TO RELATED APPLICATIONS

The present application claims the benefit of U.S. Provisional Patent Application No. 63/681,766, filed 9 Aug. 2024 and U.S. Provisional Patent Application No. 63/792,881 filed 22 Apr. 2025, which are incorporated by reference in their entirety.

### TECHNICAL FIELD

The present disclosure relates generally to systems and techniques for determining the health of a nerve during clinical applications. In particular, the disclosure describes approaches for longitudinal nerve function monitoring in clinical diagnostic and pain management contexts.

### BACKGROUND

Spinal decompression surgery is a critical intervention for patients suffering from nerve compression caused by conditions such as spinal stenosis (attributable to conditions such as herniated discs, osteophytes, or degenerative disc disease). The primary goal of spinal decompression is to alleviate pressure on the affected nerve roots, thereby reducing pain, restoring neurological function, and improving the patient's quality of life.

Spinal decompression procedures can be categorized into three main types: direct decompression, indirect decompression, and decompression with fusion. Direct or ‘non-instrumented’ decompression involves the surgeon directly removing tissue that is impinging the nerve, often by increasing the size of the spinal foramen. Indirect decompression uses devices like interspinous spacers (e.g., between adjacent posterior spinous processes) to indirectly enlarge the foramen and decompress the nerve. In cases of significant instability or when direct or indirect decompression alone is insufficient, a fusion procedure may be performed to stabilize the spine and decompress the nerve by fusing adjacent vertebrae together.

Presently, the success of certain spinal decompression procedures relies heavily on the surgeon's ability to accurately determine the degree of compression and the optimal extent of decompression required for each individual patient. For this, surgeons typically rely on their clinical judgment, experience, and visual inspection of the surgical site to guide the decompression process. This reliance on experience, and resulting procedural variability, presents legitimate risks of under-decompression, where the nerve remains partially compressed, or over-decompression, which can lead to spinal instability, dural tears, or other such complications. In either instance, the patient may not experience complete relief and may require subsequent procedures such as pain injections, revision surgeries, or even a complete fusion.

Prior to surgical intervention, patients with nerve compression conditions typically undergo conservative pain management involving therapeutic injections, medication management, and physical therapy. The management of these conditions in the clinical context presents several challenges. The subjective nature of pain assessment, coupled with the difficulty in precisely localizing the source of pain, can complicate diagnosis and treatment planning. Multiple pain generators may be present, making it difficult to identify the primary source of symptoms. Furthermore, the limited ability to objectively quantify the degree of nerve compression and the variability in treatment response among patients can lead to suboptimal outcomes.

Current diagnostic approaches rely heavily on imaging studies such as MRI or CT scans, which can identify anatomical compression but provide limited insight into the functional status of the compressed nerves. This creates a significant gap between structural findings and clinical decision-making. A nerve that appears compressed on imaging may be functionally intact, while a nerve with minimal apparent compression may exhibit significant functional impairment. This disconnect between anatomical and functional assessment can lead to unnecessary procedures or delayed interventions.

Determining the optimal timing for surgical intervention presents another significant challenge in current practice. Without objective measures of nerve function and its changes over time, decisions about when to transition from conservative management to surgical intervention often rely on subjective assessments and clinical judgment alone. This can result in prolonged patient discomfort, unnecessary procedures, or delayed surgical intervention when it would be beneficial.

The lack of objective, real-time guidance during spinal decompression surgery and the absence of quantitative methods for tracking nerve function longitudinally during clinical pain management present major challenges in optimizing patient outcomes. As such, there is a need for a reliable, evidence-based tool that can provide continuous feedback on nerve function while guiding surgical procedures and clinical care toward optimal outcomes.

### SUMMARY

The present disclosure describes a system and method for assessing nerve health during spinal surgical procedures and clinical pain management applications. The system includes a stimulator configured to deliver electrical stimuli to a nerve, a sensor configured to detect muscle responses evoked by the electrical stimuli, a display device, and a processor in communication with the stimulator, sensor, and display device.

The processor is configured to execute instructions stored in a memory to control the stimulator to deliver electrical stimuli to the nerve, determine nerve function parameters based on the muscle responses detected by the sensor, compare the determined parameters to predefined target ranges retrieved from the memory, and control the display to provide real-time feedback. The nerve function parameters may include a minimum electrical current required to evoke a detectable muscle response (stimulation threshold), a minimum electrical current required to evoke a maximal muscle response (maximal stimulation threshold), and the magnitude of the maximal muscle response.

To efficiently determine these parameters, the processor executes one or more different algorithms stored in the memory. For example, in an embodiment, a search algorithm may be executed to efficiently locate the minimum stimulation threshold, starting with an initial current range retrieved from memory and iteratively narrowing it based on detected responses. An adaptive search algorithm, leveraging data from the binary search, may then be executed to determine the maximal stimulation threshold. This approach minimizes the number of stimuli required, reducing patient discomfort and the risk of nerve fatigue.

Where applicable, predefined target ranges that are indicative of a fully decompressed nerve may be stored in and retrieved from the memory or a database to be displayed during a procedure to provide a target endpoint. Such ranges may, for example, include electrical currents of about 2 mA to 3 mA for the stimulation threshold. In some embodiments, however, the processor is configured to determine subject-specific target ranges based on individual attributes such as age, body mass index (BMI), and pre-operative neurological function assessment. In such an instance, the target ranges may include currents higher than 3 mA.

In some embodiments, the system may be integrated with a comprehensive database of historical patient data, which may be stored locally in the memory or on a remote server accessible via a network. This database may be populated over time with anonymized nerve function measurements from previous surgical procedures and clinical assessments, subject-specific attributes, and associated clinical outcomes. The processor can be configured to execute an algorithm to query this database to identify a subset of historical patients with attributes similar to the current patient. It may then define individualized target ranges based on nerve function parameters associated with successful outcomes in this subset, allowing for highly personalized patient care.

The system may further employ advanced machine learning techniques. The processor may be configured to execute a machine learning algorithm to analyze the historical patient data, identify complex patterns between pre-operative characteristics, nerve function measurements, and outcomes. Such algorithms may continuously refine predictive models used to generate individualized target ranges and risk profiles, with the processor periodically re-training the models as new outcome data is added to the database.

For clinical applications, the system provides specialized capabilities for longitudinal monitoring of nerve function across multiple clinical visits. This longitudinal tracking allows healthcare providers to monitor disease progression, assess treatment efficacy, and make data-driven decisions about the timing and necessity of surgical intervention. The system may implement normalization techniques, executed by the processor, to improve the comparability of measurements across different sessions. For example, in some embodiments, the normalization techniques may attempt to compensate for a variable separation distance between the stimulation electrode and the target nerve when contact between the stimulation electrode and nerve cannot be guaranteed.

Example position normalization approaches may include, for example, fluoroscopic landmark registration, wherein the processor executes an image processing algorithm to use anatomical landmarks to estimate electrode-nerve distance, and catheter position optimization techniques, which systematically adjust the catheter position to find the minimum stimulation threshold.

In some embodiments, the system may generate statistical risk profiles that correlate nerve function measurements with probabilities of surgical complications. These may include specific risks such as destabilization of the spine, dural tears, blood clots, cerebrospinal fluid leaks, and infections. The processor may be configured to execute an algorithm to determine a statistical risk of complications based on the nerve function parameters and subject-specific attributes retrieved from the database, and control the display to provide a clear indication of these risks.

For clinical pain management applications, the system may be integrated with existing clinical workflows. For example, in an embodiment, the system may utilize a stimulated catheter that combines nerve stimulation and therapeutic agent delivery functionalities into a single device, enabling seamless integration with interventional pain management procedures. This integration allows for real-time assessment of nerve function before, during, and after therapeutic interventions, enhancing the precision and efficacy of these procedures. In another embodiment, the system may employ a stimulation device that is particularly adapted to fit through a surgical access needle such as a 22-gauge needle.

The display may provide a graphical user interface for presenting information to the healthcare provider. It may include color-coded indicators, graphical representations of nerve function parameters relative to target ranges, and/or visual representations of risk profiles. For longitudinal monitoring, the display may present trends in nerve function parameters over time, facilitating the identification of patterns indicative of disease progression or treatment response. The interface may also allow for customization, enabling healthcare providers to focus on specific aspects they deem most relevant to a particular case.

In some embodiments, the sensor comprises a mechanical sensor with an accelerometer configured to detect a mechanical muscle response evoked by the electrical stimuli. This mechanomyography (MMG) approach allows for precise detection of muscle responses without the need for needle electrodes and while providing a significantly improved signal-to-noise ratio when compared to electromyography (EMG).

The disclosure also describes the calculation of a composite nerve function index. The processor may execute an algorithm to calculate this index as a weighted average of multiple nerve function parameters, providing a comprehensive measure of nerve health. The weighting factors for different parameters may be retrieved from memory, having been determined through empirical studies or expert consensus.

In some embodiments, the system may be integrated with data from wearable devices and mobile applications to provide objective assessment of real-world activity between clinical visits. The processor, via a communication interface, may be configured to retrieve activity data from a third-party server and execute an algorithm to correlate changes in nerve function with changes in daily activity patterns, offering additional context for interpreting nerve function measurements and assessing treatment outcomes.

Furthermore, the disclosure details various mechanisms for post-operative data collection and system improvement. After each procedure, the system can receive and incorporate outcome data into the historical patient database, which is then used by the processor to refine its predictive models. This continuous learning process allows the system to adapt and improve over time, potentially leading to better surgical outcomes and reduced complications in future procedures.

The method of operating the system includes delivering electrical stimuli to a nerve, detecting muscle responses evoked by the electrical stimuli, determining nerve function parameters based on the detected muscle responses, comparing the determined parameters to predefined or individualized target ranges, and controlling a display to provide real-time feedback to the healthcare provider.

By providing healthcare providers with objective, real-time, and personalized feedback on nerve function during both surgical procedures and clinical pain management, this technology aims to optimize treatment approaches, improve outcomes, reduce complications, and ultimately enhance patient care in the management of spinal nerve compression conditions.

### BRIEF DESCRIPTION OF THE DRAWINGS

FIG. 1 schematically illustrates an embodiment of a neural monitoring system.

FIG. 2 schematically illustrates an embodiment of a neuromuscular sensor.

FIG. 3 schematically illustrates a functional diagram of an embodiment of a control unit for a neural monitoring system.

FIG. 4 schematically illustrates an embodiment of a data visualization illustrating the longitudinal tracking of nerve health parameters for multiple nerve roots across clinical pain management visits.

FIG. 5 schematically illustrates a flow diagram of various potential integration points for a neural monitoring system within a standard pain management workflow.

FIG. 6A schematically illustrates an embodiment of a stimulated catheter with electrode configuration and therapeutic agent delivery components.

FIG. 6B schematically illustrates a cross-sectional view of the stimulated catheter of FIG. 6A, taken along the line 6B-6B.

FIG. 7A schematically illustrates an embodiment of a stimulated catheter tip design with a tip-electrode.

FIG. 7B schematically illustrates an embodiment of a stimulated catheter tip design with a predefined tip curvature.

FIG. 7C schematically illustrates an embodiment of a steerable stimulated catheter tip design with a circumferential ring electrode.

FIG. 8 schematically illustrates a segmented electrode design with independently controllable electrode segments.

FIG. 9A schematically illustrates an embodiment of a flexible multi-electrode diagnostic probe.

FIG. 9B schematically illustrates a cross-sectional view of the flexible multi-electrode diagnostic probe of FIG. 9A, taken along the line 9B-9B.

FIG. 10 schematically illustrates an anatomical diagram of a therapeutic injection within which the present neural diagnostic system may be utilized.

FIG. 11 schematically illustrates a fluoroscopic image with overlaid anatomical landmarks for electrode-nerve distance estimation.

FIG. 12 schematically illustrates a flowchart of the landmark registration and distance normalization process.

FIG. 13 schematically illustrates an embodiment of a graphical representation of stimulation thresholds recorded during a catheter position optimization procedure.

FIG. 14 schematically illustrates a flowchart of the catheter position optimization process.

FIG. 15 schematically illustrates an embodiment of a dashboard showing integration of objective nerve function data with subjective pain reports.

FIG. 16 schematically illustrates an embodiment of the data flow for predictive analytics of treatment outcomes.

FIG. 17 schematically illustrates an embodiment of a user interface displaying predictive treatment outcomes with probability estimates and confidence indicators.

FIG. 18 schematically illustrates a dashboard showing correlation between activity data and nerve function parameters.

### DETAILED DESCRIPTION

The presently described technology presents a diagnostic system that enables healthcare providers to functionally monitor the health and/or responsiveness of a compressed nerve during both surgical decompression procedures and clinical pain management applications. Through such monitoring, the present system provides spinal surgeons and pain management specialists with unique procedural insights that can be used to identify the functional status of a nerve while also reducing the risk of under-or over-decompression during surgery, optimizing pain management interventions, and/or improving overall patient outcomes. Further, in some embodiments, the system can allow for tailoring of both surgical and clinical approaches to each patient's unique anatomy and neurological condition.

Optimizing the degree of surgical decompression though intraoperative monitoring may lead to decreased complication rates, shorter hospital stays, lower healthcare costs associated with revision surgeries, prolonged recovery, and even subsequently required vertebral fusion procedures. Similarly, adequate clinical diagnostics and objective nerve function assessment may improve treatment selection/planning, optimize intervention timing, and facilitate appropriate surgical referrals. Additionally, the insights gained from the data collected by the present diagnostic system could contribute to the advancement of new spinal decompression techniques and the understanding of nerve function in various pathological conditions.

### System 10

Referring to the drawings, wherein like reference numerals are used to identify like or identical components in the various views, FIG. 1 schematically illustrates a neural monitoring system 10 that is designed to optimize spinal nerve assessment by providing a real-time functional assessment of a nerve to a user/surgeon/healthcare provider (generally referred to as a “user”). As may be appreciated, the nerve exists within an intracorporeal treatment area 12 of a subject 14 and innervates a distal muscle group of the subject 14.

In its most general sense, the system 10 comprises four main components: a stimulator 20, a neuromuscular sensor (NMS) 30, an output device (e.g., a display 40), and a host processor 50. The stimulator 20 delivers an electrical stimulus 22 to a nerve within the intracorporeal treatment area 12. The NMS 30 detects muscle responses evoked by this electrical stimulus (i.e., an “artificially induced neuromuscular response”). The display 40 provides real-time feedback to the user/surgeon/healthcare provider. The host processor 50 (also just referred to as the “processor 50”) coordinates these components, being in electrical communication with the stimulator 20, sensor 30, and display 40. In some embodiments, the display 40 and processor 50 may be integrated into a single control unit 60, which can be adapted with clips or connectors that enable the device to be mounted adjacent to the subject.

As used herein, an “artificially induced neuromuscular response” is a response of a muscle to an artificial/non-biological stimulus applied to a nerve innervating that muscle. In general, the response is “artificially induced” because the nerve is depolarized directly by the stimulus, instead of, for example, the stimulus being received through an intermediate sensory means (e.g., sight, sound, taste, smell, and touch). An example of a stimulus that may cause an “artificially-induced” muscle response may include an electrical current applied directly to the nerve or to intracorporeal tissue or fluid immediately surrounding the nerve. In such an example, if the applied electrical current is sufficiently strong and/or sufficiently close to the nerve, it may cause the nerve to involuntarily depolarize (resulting in a corresponding contraction of the muscle or muscles innervated by that nerve). Other examples of such “artificial stimuli” may involve mechanically-induced depolarization (e.g., physically stretching or compressing a nerve, such as with a tissue retractor), thermally-induced depolarization (e.g., through ultrasonic cautery), or chemically-induced depolarization (e.g., through the application of a chemical agent to the tissue surrounding the nerve).

During an artificially induced neuromuscular response, a muscle innervated by the artificially depolarized nerve may physically contract or relax (i.e., a mechanical response) and/or the electrical potential throughout the muscle may also be altered. Mechanical responses may primarily occur along a longitudinal direction of the muscle (i.e., a direction aligned with the constituent fibers of the muscle), though may further result in a respective swelling/relaxing of the muscle in a lateral direction (which may be substantially normal to the skin for most skeletal muscles). This local movement of the muscle during an artificially-induced mechanical muscle response may be measured relative to the position of the muscle when in a non-stimulated state or as a function of a sensed acceleration during the contraction/relaxation.

### Stimulator 20

As noted above, the system 10 may include one or more stimulation devices capable of selectively providing a stimulus 22 within the intracorporeal treatment area 12 of the subject 14. The system 10 may include or be configured to operate with different types of stimulation devices optimized for different clinical contexts, including surgical stimulators for intraoperative use, retractors or dilators used to establish a safe working corridor to the spine, flexible elongate stimulators for use through cannulated instruments, or stimulated catheters that can both stimulate and administer fluid therapeutic agents.

In surgical contexts, the stimulator 20 may resemble a traditional surgical instrument such as a k-wire, a ball-tip probe, a flat-tip probe, a needle, or a catheter that is intended to extend into the intracorporeal space of the subject 14. In these embodiments, the stimulator 20 includes an elongate body 24 with one or more electrodes 26 disposed on its distal end portion 28. The electrode(s) 26 may be in electrical communication with the processor 50 and may be selectively electrified by the processor 50 to provide an electrical stimulus 22 to intracorporeal tissue of the subject. In other configurations, the stimulator 20 may comprise a dilator, retractor, clip, cautery probe, pedicle screw, robotic end effector, or any other medical instrument that may be used in an invasive medical procedure.

An electrode 26 on the distal end portion 28 may comprise an electrically conductive pad or surface of the stimulator 20 that is intended to contact tissue within the intracorporeal treatment area 12 during the procedure. In some embodiments, the electrode 26 may be a distinct element, such as a gold contact that is overlaid or printed onto the body or tip of the stimulator 20. In other embodiments, the electrode 26 may simply be an uninsulated/exposed portion of the stimulator 20 that is electrically conductive and able to outwardly transmit an electrical current to surrounding tissue/fluids.

In some embodiments, the stimulator 20 may be particularly designed to access and electrically stimulate a nerve that is compressed within a spinal foramen. Such a design may include a stimulator with specialized geometry that allows the distal tip of the stimulator to extend around a portion of the spinal lamina from either an upper (superior) or lower (inferior) direction, thus enabling direct access to the nerve within the foramen.

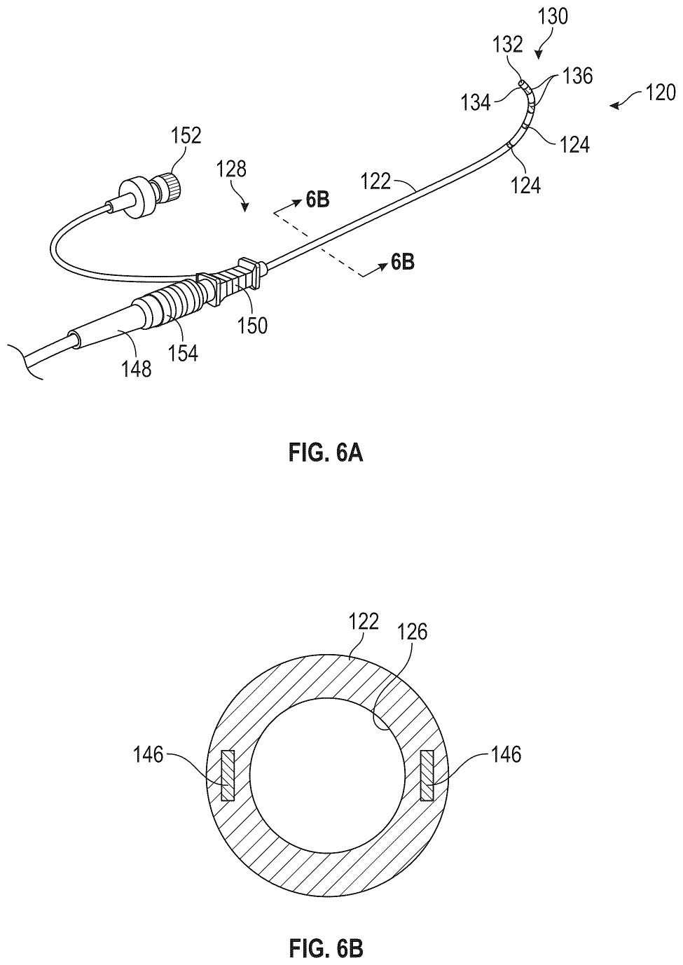

For clinical pain management applications, the system 10 may utilize a stimulated catheter, such as shown in FIG. 6, or a flexible elongate stimulator, such as shown in FIG. 9. As will be discussed in greater detail below, the flexible elongate stimulator may be configured to extend through narrow cannulated needles or transcutaneous access devices. Likewise, the stimulated catheter combines nerve stimulation and therapeutic agent delivery functionalities into a single device, facilitating integration with interventional pain management procedures such as epidural steroid injections or nerve root blocks.

Further details and embodiments of the flexible elongate stimulator and stimulated catheter construction, materials, and enhancements are provided in a later section of this disclosure.

### Neuromuscular Sensor 30

The neuromuscular sensor 30 (NMS 30) (generically referred to as the sensor 30) is the portion of the system 10 that directly contacts the subject 14 and is responsible for, at a minimum, sensing and measuring responses of the subject's muscles to the applied electrical stimulus 22 and providing a corresponding MMG output signal 32 to the processor 50. The sensor configuration may be substantially the same for both surgical and clinical applications.

A carrier material 34 is provided to operatively hold each provided sensor 30 in direct mechanical communication with the external skin surface of the subject 14. The carrier material 34 may be, for example, an adhesive pad, a pocketed patch, a cuff, and/or a sleeve that is operative to receive the sensor 30 while not substantially restricting the motion of the muscle.

In some embodiments the carrier material 34 may encapsulate and/or form a sterile barrier around the NMS 30. This may promote cost-effective reusability of the NMS 30 without subjecting it to the same sterilization requirements as if it were directly within the sterile field (i.e., absent a suitable barrier material). In some embodiments, the carrier material 34 may be a separate therapeutic or diagnostic device that is already common in surgical applications. For example, in a spinal procedure involving one or more of the L2-S1 vertebrae, it is known that nerve roots innervating the leg muscles may lie within the surgical area. During such procedures, however, compression-type anti-embolism stockings (Thrombo-Embolic-Deterrent (“TED”) hose) are typically provided around a subject's legs and feet to discourage blood clot formation. Thus, in one embodiment the carrier material 34 may be an elastic sleeve/stocking configured to apply a compressive force to the subject's leg when worn, thus eliminating the need for separate TED hose. Such a compression against the subject may present itself as an elastic tension/strain in the carrier material itself (also referred to as a “tension fit”). In surgical procedures performed higher on the spine, the carrier material 34 may include, for example, a blood pressure cuff worn around the subject's arm (or else may include functionality similar to that of a standard blood pressure cuff). In these examples, the carrier material 34 serves a function outside of that of a dedicated neuromuscular sensing device, and thus provides efficiencies in pre-op preparation and planning, while also allowing monitoring access on sometimes crowded limbs.

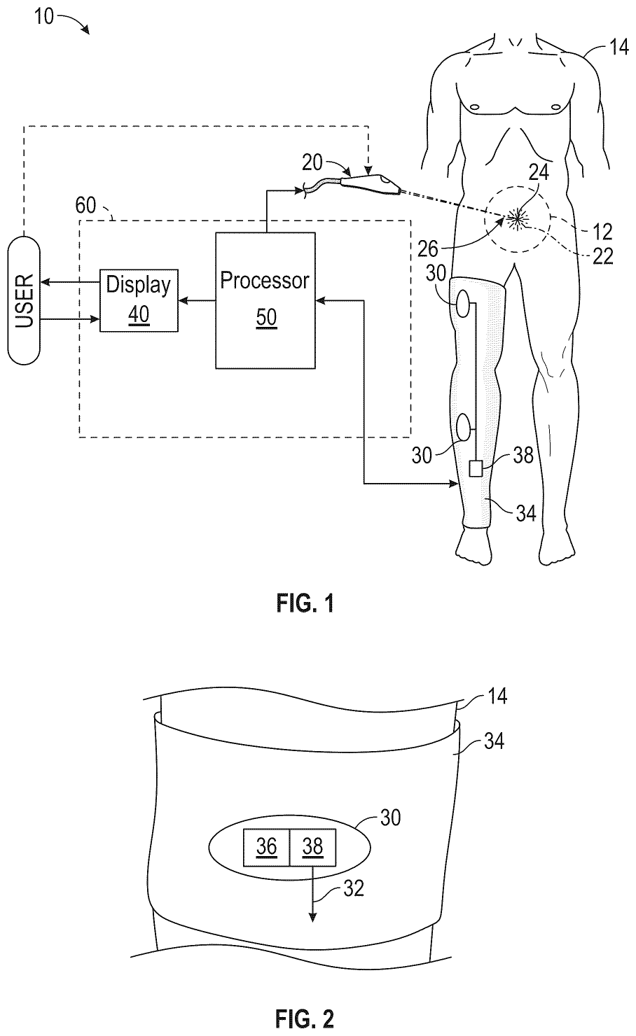

In various embodiments, such as shown in FIG. 2, each NMS 30 may comprise a mechanical sensor 36 that is operative to monitor the relative movement of the muscle that the NMS 30 is most closely coupled with. Examples of suitable mechanical sensors 36 include, strain gauges, a pressure/force transducers, position encoders, accelerometers, piezoelectric materials, or any other transducer or combination of transducers that may convert a physical motion into a variable analog or digital electrical signal. In one particular embodiment, the mechanical sensor 36 may be an accelerometer configured to detect a mechanical muscle response evoked by the electrical stimuli. In some embodiments, an NMS 30 may alternatively or additionally include one or more transdermal electrodes, needle electrodes, or other such sensors that may be operative to monitor mechanical or electrical response parameters of the subject.

In some embodiments, each sensor 30 (or collection of sensors 30) may include a local or onboard processor 38 (i.e., local to the sensor 30) that is in electrical communication with the mechanical sensor 36 of that NMS 30. The local processor 38 may be configured to, for example, pre-process and/or filter data acquired from the mechanical sensor 36 and transmit an MMG output signal 32 to the host processor 50. The MMG output signal 32 may be a digital or analog signal that is representative of the output (or filtered output) of the mechanical sensor 36. The local processor 38 may further include suitable communication circuitry to facilitate unidirectional or bidirectional digital communication with the host processor 50.

In general, processors used with the present system 10 (e.g., processors 38, 50) may be embodied as one or multiple digital computers, data processing devices, and/or digital signal processors (DSPs), which may have one or more microcontrollers or central processing units (CPUs), read only memory (ROM), random access memory (RAM), electrically-erasable programmable read only memory (EEPROM), flash memory, high-speed clocks, analog-to-digital (A/D) circuitry, digital-to-analog (D/A) circuitry, input/output (I/O) circuitry, and/or signal conditioning and buffering electronics. Any algorithms, methods, or instructions executed by a processor are understood to be stored as executable code in a non-transitory computer-readable medium, such as the memory.

### Control Unit and Data Processing

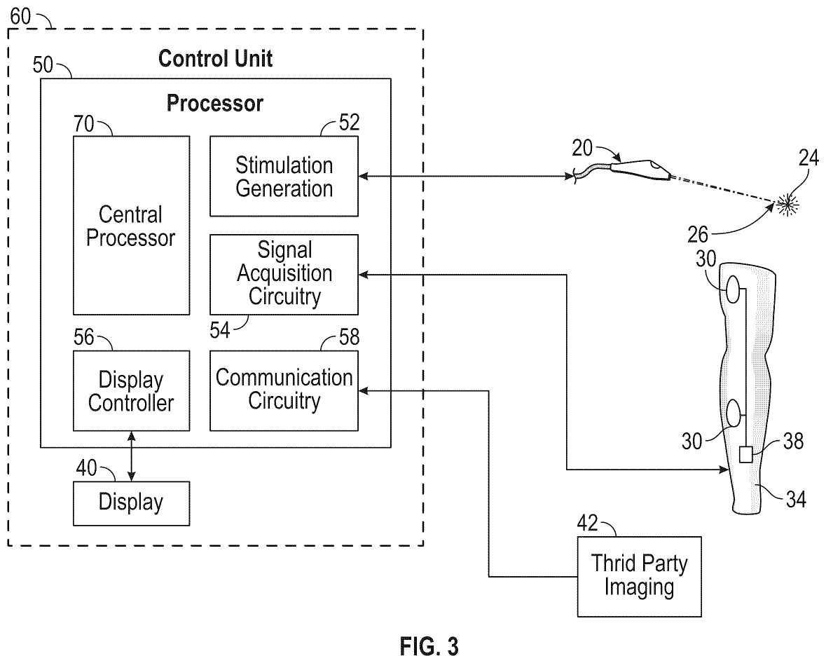

As noted above, the system 10 may include both a display 40 and a processor 50 that, in some embodiments, may be combined into a common housing or control unit 60, as shown in FIG. 3. In other embodiments, the control unit 60 may include the host processor 50 without an integrated display 40, though it may then include circuitry operative to communicate with an external display, third party device having a display, or a software application.

In a general sense, the host processor 50 is a special purpose device configured by instructions stored in an associated memory to transmit an electrical stimulus 22 to the stimulator 20, monitor the sensor 30 for the occurrence of an artificially induced neuromuscular response to the stimulus 22, determine one or more bioelectric or functional parameters of the nerve via the administered stimulus 22 and sensed response, and provide feedback about the nerve to the user/surgeon/healthcare provider via the display 40. In longitudinal monitoring applications, the processor 50 is further configured to store nerve function data in a database, which may be located in the local memory or on a remote server accessible via a network. The processor 50 executes algorithms to perform temporal analyses on the stored data and generates visualizations that facilitate the identification of trends in nerve function. It should be appreciated that for certain data processing tasks, reference to the “processor 50” is intended to cover embodiments where aspects of the data processing are performed remotely from the control unit 60 via devices in digital networked communication with a local processor.

In embodiments having an integrated display, the display 40 may include an LCD or LED-type display that is configured to provide the user/surgeon with a visual user interface. The display 40 may be capable of generating one or more visual alerts to notify the surgeon when specific nerve function thresholds are reached. These alerts may include color-coded indicators, flashing elements, pop-up notifications on the display, or graphical representation of a threshold being crossed. In some embodiments, the processor 50 may be configured to generate an audible or visual alert if a measured nerve function parameter, such as the minimum stimulation current, falls within a specified target range that is retrieved from memory and is statistically representative of the expected value of the parameter following a complete decompression. Additionally, in some embodiments, the control unit 60 may be equipped with speakers or other sound-generating devices to provide audible alerts. These audible alerts can be in the form of distinct tones, verbal announcements, or varying patterns or frequencies of sounds to indicate different stages of nerve assessment or when target ranges are achieved.

In some embodiments, the system is configured to retrieve (e.g., from memory or a remote database) and display, via the display 40, one or more target ranges for nerve-function parameters and one or more statistical profiles of risk of complications (e.g., overall and/or per-complication), the profiles being computed as functions of the measured parameter(s) and optionally individualized using subject-specific attributes. The graphical output may include, for example, visualizations of the patient-adjusted target range(s), indicators of the present measured value(s) relative to such range(s), and visualizations of outcome probabilities and complication risks that may be used intraoperatively and/or pre-operatively. U.S. Pat. No. 12,279,880 (titled “System and Method for Assessing Nerve Health During a Spinal Decompression Procedure”) is hereby incorporated by reference in its entirety and for all that it discloses. In particular, the '880 Patent provides examples of generating and presenting individualized target ranges and complication-risk visualizations, including outcome-adjusted and risk-adjusted ranges, surgeon-selectable constituent risks, and combined displays of ranges and risk curves.

Referring to FIG. 3, in some embodiments, host processor 50 may include one or more functional units or modules that provide specialized functionality or capabilities to the processor 50. Each functional unit or module may comprise the requisite hardware, circuitry, software, firmware, or combinations thereof to perform the intended function. Such modules may include, for example, a stimulation generator 52, signal acquisition circuitry 54, a display controller 56, and communications circuitry 58. Each of these functional units may operate at the direction and coordination of a supervising/central processing unit 70 (a CPU 70) that is in bidirectional communication with each module. While FIG. 3 schematically illustrates each module as being separate and distinct from other modules or the CPU, it should be understood that some or all modules may be implemented via a common integrated microcontroller or application-specific integrated circuit (ASIC). Further, in some embodiments the modules as illustrated may simply be representative of distinct functionality or software capabilities of the host processor 50, defined by instructions stored in memory.

The stimulation generator 52 may be operative to generate a current-controlled electrical stimulus 22 that can be transmitted to an electrode 26 on the distal end of the stimulator 20. This electrical stimulus 22 may subsequently be administered to the intracorporeal tissue of the subject from the stimulator/electrode and returned to the unit via an adjacent electrode or via an associated ground patch that is adhered to the subject.

The electrical stimulus 22 generated by the stimulus generator 52 may, for example, be a periodic stimulus that includes a plurality of sequential discrete pulses (e.g., a step pulse) provided at a frequency of less than about 20 Hz, or between about 2 Hz and about 16 Hz. Each pulse may have a pulse width within the range of about 50 μs to about 400 μs. In other examples, each discrete pulse may have a pulse width within the range of about 50 μs to about 200 μs, or within the range of about 75 μs to about 125 μs. Additionally, in some embodiments, the current amplitude of each pulse may be independently controllable by the processor 50.

In some embodiments, the processor 50 may execute an algorithm that automatically adjusts the stimulation parameters, such as pulse width and frequency, based on the patient's real-time response and the stage of the procedure. For instance, the processor 50 may retrieve initial parameters of a 200 μs pulse width and a 4 Hz frequency from memory. If the elicited muscle response is weak or inconsistent, the processor 50 may automatically increase the pulse width to 300 μs to improve the sensitivity of the neuromonitoring. Conversely, if the muscle response is consistently strong, the system may reduce the pulse width to minimize the risk of nerve fatigue or injury. This adaptive approach may serve to optimize the stimulation parameters throughout the procedure, thus maintaining the reliability and specificity of the neuromonitoring.

If a nerve extends within a predetermined distance of the electrode 26, the electrical stimulus 22 may cause the nerve to depolarize, resulting in a mechanical twitch of a muscle that is innervated by the nerve (i.e., an artificially-induced mechanical muscle response). As noted above, each NMS 30 may be specially configured to monitor a local mechanical movement of an adjacent muscle group of the subject 14. In response to this sensed movement, each respective mechanical sensor 36 may generate a digital or analog mechanomyography (MMG) output signal 32 that corresponds to the sensed mechanical movement, force, and/or response of the adjacent muscle. The signal acquisition circuitry 54 of the processor 50 may be adapted to receive this MMG output signal 32 from the NMS 30, digitize it, condition and/or filter (if desired) it and make it available in memory for subsequent processing by the CPU 70.

The display controller 56 may include any required memory or graphical processing units (GPUs) required to supply a display signal to the display 40. The communications circuitry 58 may include any required circuitry, wi-fi communication circuitry, BLUETOOTH communication circuitry, ethernet communication circuitry, modems, USB-C data transmission circuitry, wireless radio antennas, cellular communication chipsets, and/or any associated code to facilitate unidirectional or bidirectional digital communication between the processor 50 and external sources via either a wired or wireless communications protocol.

In some embodiments, the neural monitoring system 10 and/or display controller 56 may be integrated with imaging modalities, such as fluoroscopy or ultrasound (i.e., “third party imaging” 42). In these configurations, the processor 50 may receive, via its communication circuitry 58 or another input, an imaging signal representative of an image or video, and the display controller 56 may then output the imaging from these external third-party imaging modalities 42 via the display 40. Integration of such third-party imaging modalities 42 may allow the healthcare provider to visualize the anatomical structures in real-time while simultaneously monitoring the nerve function. For example, the healthcare provider may use fluoroscopy to guide the placement of the stimulation probe or catheter and ensure that the electrode is in proximity to the target nerve root. The fluoroscopic images, via the fluoroscopy device/system may be received and displayed on the same screen as the neuromonitoring data, thus providing a comprehensive view of the treatment area. Similarly, ultrasound imaging may be used to assess the extent of decompression or nerve impingement. The ultrasound images can be correlated with the nerve function measurements, enabling the healthcare provider to make targeted adjustments based on both anatomical and functional information.

For longitudinal monitoring in clinical applications, the processor 50 may include or be in digital communication with a database having enhanced data storage capabilities to retain nerve function measurements across multiple clinical visits. The processor 50 may implement and/or interface with a database structure that associates each measurement with metadata such as, for example, a unique patient identifier, the date of measurement, the procedure performed, the healthcare provider, stimulation parameters, and/or patient-reported outcomes. This database structure facilitates the retrieval and analysis of historical measurements for comparison with current data.

The processor 50 (e.g., via the CPU 70) may execute resident software, firmware, or embedded processing routines that are operative to analyze the output from the neuromuscular sensors 30 and identify muscle responses that were induced by an electrical stimulus 22 applied via the stimulator 20 (i.e., an artificially induced response). More specifically, these detection/identification algorithms stored in memory may attempt to establish, with a high degree of confidence, that a detected muscle movement is the result of a nerve being artificially depolarized (i.e., via a stimulus 22 administered by the stimulator 20) and not simply a subject-intended muscle movement, an environmentally caused movement (e.g., bumping the operating table), or an artifact of another aspect of the procedure (e.g., sequential compression devices or cautery). In one example, such an algorithm is described in detail in U.S. Pat. No. 11,980,476, which is incorporated by reference in its entirety.

In varying embodiments, the detection algorithms may be performed in the analog/time domain, the digital/frequency domain, and/or may employ one or more wavelet analyses or multi-stage analysis techniques. Additional techniques such as response gating, stimulus frequency modulation, artificial intelligence/structured machine learning, and/or ensemble approaches may also be used to make this detection more robust and/or provide a greater degree of confidence in the detection. Examples of such techniques are further described in U.S. Pat. No. 8,343,065, issued on Jan. 1, 2013, and US Patent Application Publication No. 2015/0051506 (filed on Aug. 13, 2013) both of which are hereby incorporated by reference in their entirety.

### Nerve Function Parameters

### Core Parameter Definitions

To effectively monitor nerve function and guide both surgical procedures and clinical pain management, the present neural monitoring system 10 is configured to determine one or more functional parameters of the nerve (i.e., “nerve function parameters,” also referred to as “nerve health parameters”) through the performance and/or execution of one or more algorithms stored in memory associated with the processor 50. In a general sense, these algorithms cause the processor 50 to direct the administration of a controlled electrical stimulus 22 and then analyze the MMG output signals 32 generated as a result of the stimulus/stimulation to understand the functional status of the nerve. Examples of functional parameters that may be determined in this manner include one or more of: the minimum stimulation current that is required to elicit a detectable, threshold response in the muscle (i.e., the “stimulation threshold”); the minimum stimulation current that is required to elicit a maximal response of the muscle (i.e., the “saturation threshold”); or the magnitude of the response of the muscle during a maximal contraction (i.e., “the maximum muscle output”).

In some embodiments, the processor 50 is configured to provide real-time feedback on nerve function by executing an algorithm to track changes in functional parameters throughout the surgical procedure or across multiple clinical visits. This enables healthcare providers to make informed decisions about the extent of intervention needed and helps avoid suboptimal treatments. In doing so, the system 10 may help identify potential issues or unrecognized nerve impingements before concluding the procedure. For instance, if parameters fail to improve as expected or plateau above a predefined target endpoint or range retrieved from memory, the processor 50 may control the display 40 to alert the healthcare provider to the possibility of residual impairment. These insights may allow or encourage healthcare providers to further investigate, using techniques like imaging or direct visualization, to identify and/or address any remaining compression sources. By facilitating more complete and effective assessment through functional diagnostics, the system may reduce the risk of persistent symptoms and the need for revision procedures.

The determination of one or more of these functional parameters via the present system 10 provides objective criteria for assessing nerve function intraoperatively or in clinical settings. By executing an algorithm to compare the parameter values to established normative ranges or patient-specific targets retrieved from a database, the processor 50 can provide a clear indication of whether the intervention has achieved its intended goals or whether further measures may be warranted.

The following describes examples of three functional parameters that may be used to quantitatively assess a nerve and provide evidence of neural compression:

### Minimum Stimulation Current for Evoking Muscle Response (“Stimulation Threshold”)

The minimum stimulation current required to evoke a muscle response, also referred to as the “stimulation threshold” or “minimum stimulation threshold” is the lowest electrical current intensity required to evoke a detectable or threshold-level muscle response in a muscle that is innervated by the nerve. Because the physical distance between the electrode and the nerve impacts this threshold (i.e., a larger current is required as the distance increases), to reduce variability, it is preferable for this measure to be taken when the electrode that is providing the stimulus is in close proximity to the nerve. This parameter is a measure of nerve excitability and reflects the ease with which the nerve can be stimulated to generate an action potential.

In the context of spinal decompression and clinical assessment, monitoring the stimulation threshold provides valuable information about the functional status of the nerve. A high stimulation threshold suggests that the nerve is less excitable, possibly due to the mechanical pressure exerted by the surrounding tissues. As decompression or treatment progresses and the pressure on the nerve is relieved, the stimulation threshold is expected to decrease, indicating improved nerve excitability.

The neural monitoring system 10 determines the stimulation threshold by having the processor 50 execute an algorithm to systematically vary the intensity of the electrical stimuli 22 delivered through the stimulator 20 (i.e., with the electrode in close proximity to the nerve). The processor 50 then analyzes the MMG output signals 32 to identify the minimum current intensity that consistently evokes a muscle response above a predefined amplitude threshold retrieved from memory.

By executing an algorithm to track changes in the stimulation threshold over the course of a procedure or across multiple clinical visits, the system 10 can provide the healthcare provider with real-time feedback on the effectiveness of interventions in restoring nerve excitability. A significant decrease in the stimulation threshold compared to a baseline is generally considered a positive sign, indicating improved nerve function. Likewise, the stimulation threshold falling within a predefined target range of current values can provide an indication that the nerve is no longer compressed. For clarity, the terms “minimum stimulation current” and “stimulation threshold” are used interchangeably throughout this specification to refer to the lowest electrical current intensity required to evoke a detectable muscle response.

### Minimum Stimulation Current for Maximal Muscle Response (Saturation Threshold)

The minimum stimulation current required to elicit a maximal response of the muscle, referred to as the “maximal stimulation threshold” or “saturation threshold,” refers to the minimum electrical current intensity that is required to evoke the maximum achievable muscle response in a muscle that is innervated by that nerve. Like the stimulation threshold described above, this parameter is also influenced by the distance between the electrode and the nerve. Therefore, to remove variability, it is preferable for the saturation threshold to be determined when the electrode administering the stimulus is in close proximity to the nerve.

The saturation threshold parameter provides information about the overall functional capacity of the nerve-muscle system and the number of motor units that can be recruited by the electrical stimulus. The saturation threshold generally represents the minimum electrical current level at which all available motor units in the muscle are recruited and depolarized, thus providing a measure of the overall functional capacity of the nerve-muscle system. During compression, the current required for a maximal response may be elevated due to impaired nerve conduction or reduced responsiveness of the muscle.

In the context of nerve assessment, monitoring the saturation threshold (and changes in the threshold) can provide insights into the extent of acute or chronic nerve damage and the potential for functional recovery. A compressed nerve may exhibit a higher saturation threshold compared to a healthy nerve, suggesting that a larger proportion of motor units are not functioning properly, have decreased sensitivity, or require a greater stimulus current to be recruited. As intervention progresses, this threshold is expected to decrease, indicating improved nerve function and muscle recruitment. The processor 50 therefore may be configured to execute an algorithm to track changes in the saturation threshold in an effort to provide additional guidance to the healthcare provider regarding the nerve function.

The neural monitoring system 10 may determine the saturation threshold by having the processor 50 execute an algorithm to incrementally increase the intensity of the electrical stimuli 22 beyond the stimulation threshold until the amplitude of the MMG output signal 32 reaches a plateau. This plateau indicates that all available motor units are being recruited and that further increases in stimulation intensity do not result in a larger muscle response. Following this, the lowest current that elicits this plateau response is regarded as the maximal threshold. In some embodiments, the saturation threshold may be artificially defined as a current that induces a response that is a predefined percentage less than the magnitude of a full maximal response (e.g., 95% of the maximum achievable muscle response, or 90% of the maximum achievable muscle response, or even a percentage selected from the range of about 50% to about 100% of the maximum achievable muscle response).

In some embodiments, the processor 50 may be configured to execute an algorithm to determine the saturation threshold by administering a plurality of pulses that each have a current intensity below the threshold. In doing so, the processor 50 may record a plurality of data points in memory, where each data point includes a stimulus current value and an MMG output signal value. To then determine the threshold, the processor 50 may fit a curve through the recorded [stimulus current, MMG output signal] pairs and extrapolate this curve toward the threshold. (i.e., where the slope of the curve should decay toward zero as the current intensity is increased toward the threshold). In another embodiment, the processor 50 may execute an algorithm to systematically increase the current level until multiple responses are achieved within a predefined error margin of each other (i.e., the plateau).

By comparing the saturation threshold before and after intervention, the system can assess the extent of functional recovery achieved by the procedure. A significant decrease in the saturation threshold, accompanied by an increase in the magnitude of the muscle response, is generally considered a positive outcome, suggesting improved nerve function.

### Magnitude of a Maximal Muscle Response

The magnitude of a maximal muscle response refers to the maximum strength or physical response of the muscle to a maximal or supramaximal electrical stimulus administered to the nerve innervating that muscle. Unlike the prior two parameters, maximal response magnitude or maximum muscle output is not affected by the distance between the electrode and the nerve as it is simply testing the maximum possible output from the nerve-muscle system and reflects the number and synchronization of motor units activated by the stimulus.

The magnitude of the maximal muscle response may be measured or quantified by the processor 50 as one or more of: the maximum amplitude of the MMG output signal; the peak-to-peak amplitude of the MMG output signal; the root-mean-squared amplitude of the MMG output signal; or an area under the curve of the MMG output signal. This parameter provides valuable information about the strength and overall functional capacity of the nerve-muscle system. During compression, the magnitude of the maximal response may be diminished due to impaired nerve conduction and reduced motor unit recruitment. As treatment progresses and the nerve function improves, the magnitude of the muscle response to a given stimulus is expected to increase, thus reflecting the restored ability of the nerve to effectively activate the muscle. This increase in muscle response magnitude is often accompanied by a decrease in the stimulation and saturation thresholds, suggesting a comprehensive improvement in nerve function. The processor 50 therefore may be configured to execute an algorithm to monitor changes in the maximal response magnitude to provide additional feedback to the healthcare provider regarding the effectiveness of intervention.

The neural monitoring system 10 determines the magnitude of the muscle response by having the processor 50 direct the delivery of an electrical stimulus having a stimulation current that is at a known level at or above the saturation threshold. It then analyzes the MMG output signal to determine a peak response, an average response, or a power metric (e.g., the area under the curve of the MMG output signal 32) in response to that electrical stimulus. In certain scenarios, this parameter may be particularly valuable because the stimulation current can be selected at a level that is known to be greater than the saturation threshold, and thus direct contact between the electrode and nerve is not strictly required.

Another example of a nerve function parameter may include response latency (i.e., time from stimulus to mechanical muscle response onset).

### Determination Methods

In one embodiment, the processor 50 may execute a multi-stage algorithm stored in memory to efficiently determine multiple functional parameters of the nerve while minimizing the total number of discrete stimuli required. This approach consists of two main stages:
Stage 1: A binary search algorithm is executed by the processor 50 to efficiently locate the minimum stimulation threshold.
Stage 2: An adaptive search algorithm is executed by the processor 50 to efficiently determine the saturation threshold by leveraging information from Stage 1.

By leveraging the data collected in the first stage to inform the next, the system reduces redundant stimuli and makes intelligent selections for stimulus currents in Stage 2. This approach not only saves time but also minimizes patient discomfort and the risk of nerve fatigue or injury from excessive stimulation. Furthermore, the efficiency and speed of the testing procedure enables repeated assessments throughout the intervention process while providing minimal disruption to the overall procedure.

During the binary search algorithm (Stage 1), the processor 50 begins by retrieving a working range of stimulus intensities from memory, typically 0-20 mA, which encompasses the expected minimum stimulation thresholds for both healthy and compressed nerves. The processor 50 then directs the stimulation generator 52 to generate an electrical stimulus 22 having a current at the midpoint of this range (e.g., 10 mA). This stimulus 22 is then transmitted to the nerve while the processor 50 simultaneously monitors for an evoked muscle response via the NMS 30 and signal acquisition circuitry 54.

If a response is detected, the processor 50 narrows the search range to the lower half of the previous range (e.g., 0-10 mA) and repeats the process, stimulating with a current at the new midpoint. Conversely, if no response is detected, the processor narrows the search range to the upper half (e.g., 10-20 mA). This process of halving the search range and stimulating at the midpoint current level continues until the minimum stimulation threshold is determined within a desired resolution, typically less than 1 mA.

After determining the minimum stimulation threshold in Stage 1, the processor 50 proceeds to find the maximal stimulation threshold via a Stage 2 detection. The processor 50 may execute an algorithm to analyze the stimulus-response relationship observed during Stage 1, including the rate of change in response magnitude, the determined minimum stimulation threshold, and the shape of the stimulus-response curve at currents above the minimum threshold.

Based on this analysis, the processor 50 may employ a predictive algorithm to estimate the likely range for the maximal threshold and implement an adaptive binary search within this predicted range. The search may terminate when the increase in response magnitude between adjacent test points falls below a predefined threshold (e.g., 5%) or when the search range narrows to a predefined resolution (e.g., less than about 2 mA).

Once the maximal stimulation threshold has been determined, the processor 50 may measure and record the magnitude of the evoked muscle response to a stimulus having a current magnitude at (or above) the maximal stimulation threshold. This response magnitude may be quantified using one of the methods described previously.

An alternate manner of determining the minimum stimulation threshold is described in U.S. patent application Ser. No. 19/279,814, filed on 24 Jul. 2025, which is incorporated by reference in its entirety.

### Significance of Changing Nerve Function Parameters Over Time

The nerve function parameters described above hold significant importance when tracked over extended periods, particularly in clinical applications. Changes in these parameters over time can provide valuable insights into disease progression, treatment response, and optimal timing for surgical intervention.

For disease progression monitoring, the processor 50 can execute an algorithm to track the evolution of nerve function parameters across multiple clinical visits. A progressive increase in stimulation threshold or saturation threshold, or a decrease in maximal response magnitude, may indicate worsening nerve compression or deteriorating nerve function. This longitudinal data, stored in a database, may reveal patterns that are not apparent from single-time-point assessments, such as seasonal variations, activity-related changes, or gradual deterioration that might otherwise go unnoticed.

The temporal tracking of nerve function parameters also enables objective assessment of treatment response. Following therapeutic interventions such as epidural steroid injections, physical therapy, or medication adjustments, changes in nerve function parameters can provide quantitative evidence of efficacy. Improvement in parameters following intervention suggests a positive response, while lack of improvement may indicate the need for alternative approaches. This objective feedback can guide treatment refinement and help healthcare providers make data-driven decisions about continuing or modifying current management strategies.

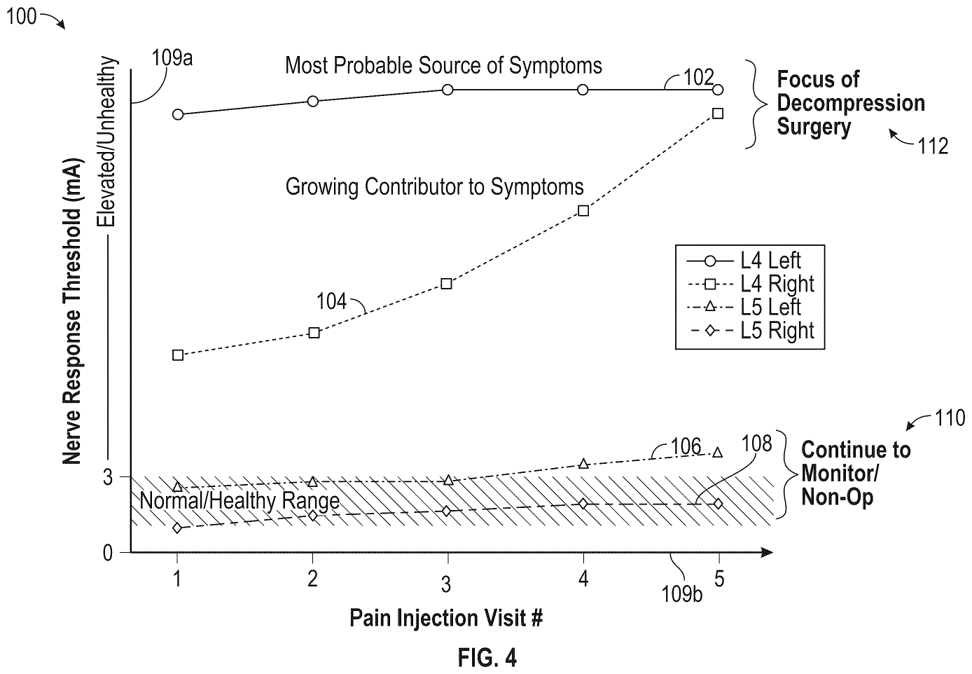

Perhaps most significantly, longitudinal tracking of nerve function parameters can inform optimal timing for surgical intervention. As illustrated in the graph 100 of FIG. 4, the processor 50 can be configured to monitor multiple nerve roots (e.g., L4 left 102, L4 right 104, L5 left 106, and L5 right 108) over time, tracking their respective stimulation thresholds across multiple pre-operative pain management visits. Patterns in this data, such as persistently elevated thresholds despite conservative treatment (e.g., L4 left 102) or progressively worsening thresholds (e.g., L4 right 104), may indicate the need for surgical intervention. Conversely, nerve roots showing stable, near-normal thresholds (e.g., L5 left 106 and L5 right 108) may not require surgical attention.

The processor 50 may execute algorithms that analyze these longitudinal trends to generate direct or indirect recommendations regarding surgical timing. These algorithms may consider factors such as the rate of parameter change, the persistence of abnormal values, the response to conservative treatments, and the correlation with patient-reported symptoms. By providing objective, quantitative data on nerve function changes over time, the system 10 helps bridge the gap between initial symptom presentation and surgical decision-making, potentially reducing unnecessary procedures and facilitating timely intervention when warranted.

### Clinical Applications Framework

### Technical Challenge in Clinical Assessment

The diagnosis and treatment of spinal nerve compression presents a fundamental technical challenge in modern medicine, particularly in clinical pain management applications. This diagnostic challenge is rooted in the complex nature of nerve innervation patterns, where multiple nerve roots often contribute to the function of individual muscle groups, making it difficult to isolate specific sources of functional impairment through traditional examination methods. While modern imaging techniques such as MRI can identify anatomical nerve compression, they provide limited insight into the functional status of compressed nerves, creating a significant gap between structural findings and clinical decision-making.

Current pain management practices for conditions such as radiculopathy and spinal stenosis face several challenges. The subjective nature of pain assessment, coupled with the difficulty in precisely localizing the source of pain, often complicates diagnosis and treatment planning. Furthermore, the limited ability to objectively quantify the degree of nerve compression and the variability in treatment response among patients can lead to suboptimal outcomes. Determining the optimal timing for surgical intervention also presents a significant challenge in current practice. These limitations collectively contribute to the potential for unnecessary procedures and prolonged patient discomfort.

This diagnostic uncertainty particularly impacts clinical pain management, where patients typically undergo multiple therapeutic procedures over months of treatment before potential surgical intervention. During these routine procedures, physicians often access target nerve roots using fluoroscopic guidance to deliver therapeutic agents. However, these interventions currently yield no quantitative data about nerve function for meaningful comparison across treatments, representing a significant missed opportunity for objective assessment.

The nerve function assessment system provided herein can address many of these challenges by providing objective, quantifiable data on nerve function. This information assists healthcare providers in identifying specific compressed nerve roots, quantifying the degree of functional impairment, and tracking changes in nerve function over time. Such data can inform treatment planning and optimization, as well as guide decisions about the timing and necessity of surgical interventions. This system can therefore serve as a powerful tool to inform healthcare providers, including doctors, surgeons, and physical therapists about the functional status and trends of the affected nerves. This objective data can be used to complement clinical judgment and other diagnostic information, enabling more informed decision-making throughout the pain management process.

### Integration With Existing Pain Management Workflow

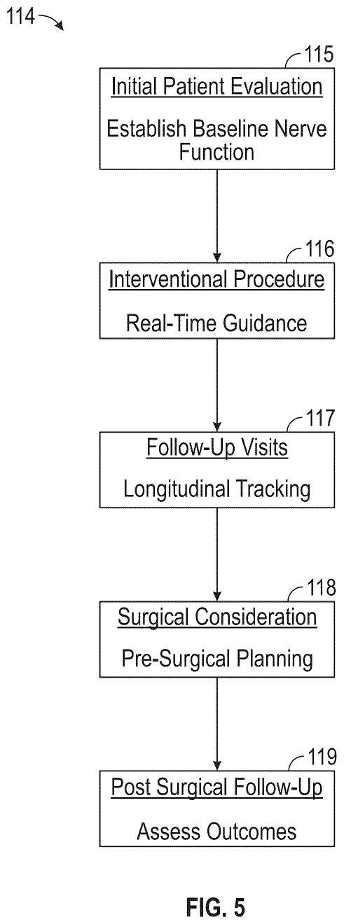

The nerve function assessment system 10 may be designed to seamlessly integrate into existing pain management workflows, thus enhancing rather than disrupting current practices. As illustrated in FIG. 5, the system 10 may be incorporated at multiple points throughout the continuum of care 114 for patients with spinal nerve compression conditions.

During initial patient evaluations 115, the system 10 can be used to establish baseline nerve function parameters. This initial assessment, typically performed in conjunction with a comprehensive physical examination and review of imaging studies, provides objective quantification of nerve function at presentation. The processor 50 may store this baseline measurement in a database, associated with a unique patient identifier, where it serves as a reference point for tracking changes over time and evaluating the effectiveness of subsequent interventions. By incorporating nerve function assessment into the initial evaluation, healthcare providers can obtain a more complete understanding of the patient's condition, potentially improving diagnostic accuracy and initial treatment selection.

For interventional procedures 116 such as epidural steroid injections or nerve root blocks, the system 10 may provide real-time information on nerve function before, during, and after the intervention. Prior to administering the therapeutic agent, the healthcare provider can use the system 10 to locate the target nerve and obtain pre-intervention nerve function measurements. This not only assists in precise targeting but also provides a pre-treatment baseline specific to that session. During the intervention, the system 10 can provide continuous feedback on electrode position relative to the nerve, potentially enhancing the precision of agent delivery. Following the intervention, the processor 50 can determine additional nerve function measurements to assess the immediate impact on nerve function, providing objective evidence of technical success beyond subjective patient feedback.

In follow-up visits 117, regular nerve function assessments can be incorporated to objectively track changes in nerve status over time. These follow-up measurements, when compared by the processor 50 to the baseline and previous measurements retrieved from the database, can reveal trends in nerve function that may not be apparent from the patient's subjective reports alone. Such objective tracking can help distinguish between temporary fluctuations and significant changes in nerve function, informing decisions about treatment continuation, modification, or escalation.

For patients considering surgical intervention 118, the system 10 offers detailed information on nerve function to aid in surgical planning and patient counseling. Comprehensive assessment of multiple nerve roots, as shown in FIG. 4, can help identify which specific nerves would benefit from surgical decompression. This information may guide the surgeon in determining the appropriate levels for intervention and the most suitable surgical approach. Additionally, the longitudinal data collected through clinical monitoring can provide insights into the natural history of the patient's condition, informing discussions about the potential benefits and timing of surgery.

In post-surgical follow-up 119, the system 10 can be used to objectively assess the functional outcomes of the intervention. By having the processor 50 compare post-operative nerve function parameters with pre-operative measurements retrieved from the database, healthcare providers can evaluate the effectiveness of the surgery in improving nerve function, independent of subjective patient reports. This objective assessment may help identify residual or recurrent nerve compression, guiding decisions about additional interventions if necessary.

By providing objective data at these key points throughout the pain management workflow, the system 10 supports healthcare providers in making more informed treatment decisions without replacing their clinical expertise. The integration of nerve function assessment into routine pain management procedures transforms these interventions from purely therapeutic events into valuable diagnostic opportunities, enhancing the overall quality of care while maintaining workflow efficiency.

### Unique Challenges in Clinical Context

The technical problem in obtaining consistent nerve function measurements during clinical procedures involves multiple interrelated challenges that distinguish this context from intraoperative monitoring. Perhaps the most significant challenge is the variable electrode-nerve distance that exists during clinical procedures. Unlike intraoperative monitoring, where direct visualization and contact with the nerve is possible, clinical procedures must rely on indirect approaches where the stimulation electrode cannot be guaranteed to maintain consistent contact with the target nerve. Research and empirical data suggest a relationship may exist between electrode-nerve separation distance and the stimulation threshold required to evoke a muscle response. For example, in some cases, for every approximately 1 mm increase in separation distance between the electrode and nerve, there may be an approximate 1 mA increase in the stimulation threshold required to depolarize the nerve. This distance-dependent relationship introduces variability in measurements, complicating the comparison of stimulation thresholds between different sessions or procedures.

Related to the variable electrode-nerve distance is the challenge of non-visual placement of the stimulator or catheter. In clinical contexts, the nerve is typically not directly visible, and the healthcare provider must rely on indirect methods such as fluoroscopic guidance, anatomical landmarks, or patient feedback to position the stimulator. Without direct visualization, confirming the precise relationship between the electrode and the target nerve becomes difficult, introducing potential variability in measurement conditions between sessions.

The potential need for normalization across sessions presents a challenge in clinical nerve assessment. To derive meaningful insights from longitudinal data, measurements taken at different times and potentially by different healthcare providers must be comparable. Without appropriate normalization techniques executed by the processor 50, variations in electrode placement, patient positioning, or procedural conditions may confound the interpretation of changes in nerve function parameters over time.

Data comparability requirements extend beyond normalization to encompass standardization of measurement protocols, stimulation parameters, and response detection thresholds. In a clinical context, where measurements may be taken weeks or months apart, maintaining consistent measurement conditions becomes increasingly important for valid comparisons. Yet, the dynamic nature of clinical practice and the potential involvement of multiple healthcare providers introduce variability that must be systematically addressed.

Integration with existing procedures presents both a challenge and an opportunity. Clinical nerve assessment must be performed without significantly disrupting or prolonging established clinical workflows. The assessment should ideally be incorporated into routine procedures, such as diagnostic blocks or therapeutic injections, without requiring substantial additional time or resources. This integration requires careful consideration of procedure duration, additional equipment needs, and healthcare provider training.

Collectively, these challenges highlight the need for specialized approaches and technologies for clinical nerve assessment that differ from those used in intraoperative monitoring. The following sections describe various embodiments and solutions to these challenges, including position normalization techniques and stimulator designs, which can provide for more reliable and consistent nerve function assessment in the clinical setting.

### Longitudinal Tracking Requirements

Effective longitudinal tracking of nerve function in clinical applications desirably leverages a framework that promotes measurement consistency, data integrity, and meaningful interpretation over time. This framework may address several key requirements to enable reliable tracking of nerve function across multiple clinical visits spanning weeks or months.

A fundamental requirement is the need for consistent measurements over time, despite inevitable variations in measurement conditions. To provide improved consistency, the processor 50 may execute algorithms to minimize or account for sources of variability such as electrode placement, patient positioning, and procedural differences between sessions. While described in greater detail below, position normalization approaches may be employed to address the variable electrode-nerve distance, while standardized protocols for stimulation parameters and response detection may help ensure measurement consistency irrespective of the specific healthcare provider performing the assessment.

The system 10 may further incorporate a database structure, which may be a relational or non-relational database stored locally or on a remote server, that organizes nerve function measurements and associated metadata in a manner that facilitates temporal analysis and comparison. The processor 50 may implement a data processing pipeline that includes storage of raw measurement data, execution of normalization procedures, and execution of analytical methods for identifying trends and patterns in the longitudinal data.

Within the database, each nerve function measurement may be associated with comprehensive metadata that includes, for example, the date and time of measurement, the specific nerve assessed, the procedure performed, the healthcare provider, the stimulation parameters used, the position normalization approach employed, and any relevant patient feedback or observations. Such metadata may enables appropriate grouping and filtering of measurements for comparison and analysis, while also providing context for interpreting apparent changes in nerve function.

In some embodiments, the database may implement a hierarchical data structure that organizes measurements by patient, nerve, and session, allowing for multi-level analysis of trends. This structure may support the visualization of longitudinal data at different scales, from detailed session-by-session comparisons to broader trend analysis across months of treatment.

To support integration with clinical decision-making, the processor 50 may incorporate visualization tools that present longitudinal trends in an intuitive and actionable format. These visualizations, such as the multi-nerve tracking graph 100 shown in FIG. 4, may help healthcare providers identify patterns, recognize significant changes, and make informed decisions about treatment continuation, modification, or escalation to surgical intervention.

By addressing these requirements for longitudinal tracking, the system 10 transforms discrete nerve function measurements into a continuous record of nerve health that can inform clinical decision-making throughout the course of treatment. This longitudinal perspective provides insights that would not be apparent from isolated measurements, potentially enhancing the precision and effectiveness of clinical care for patients with spinal nerve compression.

### Clinical Stimulator Design Embodiments

### Stimulated Catheter

In some embodiments of the clinical nerve assessment system, a stimulated catheter 120 may be provided that integrates nerve stimulation and therapeutic agent delivery functionalities into a single device. The catheter 120 may be optimized for clinical pain management applications and may be configured to be compatible with standard fluoroscopic guidance techniques while providing reliable nerve stimulation for functional assessment.

Referring to FIG. 6A-6B, the stimulated catheter 120 may comprise an elongate tubular body 122 with overall dimensions selected to be appropriate for the intended anatomical targets. For epidural and nerve root applications, the catheter 120 may have an outer diameter between approximately 12-20 gauge (approximately 2.77-0.91 mm) and a length between approximately 50-150 cm, which may be selected based on the specific application, access device (if used), and patient anatomy.

The tubular body 122 of the catheter 120 may be constructed from one or more medical-grade materials such as polyurethane, polyethylene, nylon, or combinations thereof. These materials may be selected based on properties including, but not limited to, biocompatibility, flexibility, and visibility under fluoroscopy. In some embodiments, the catheter 120 may include one or more radiopaque markers 124 incorporated at strategic locations along the tubular body 122 to enhance visualization under imaging techniques such as fluoroscopy.

As shown in the cross-sectional view of FIG. 6B, the catheter 120 may include a central lumen 126 extending from a proximal end 128 to a distal end 130 of the tubular body 122. The central lumen 126 may have an inner diameter between approximately 0.2-0.8 mm, which may be selected to be appropriate for delivering therapeutic agents such as local anesthetics, steroids, or other pain management medications. The central lumen 126 may terminate in one or more exit ports 132 located at or near the distal end 130 of the catheter 120.

For nerve stimulation functionality, the catheter 120 may incorporate one or more electrodes at or near the distal end 130. In one embodiment, as illustrated in FIG. 6A, the catheter 120 may include a distal tip electrode 134 and one or more ring electrodes 136 positioned proximal to the distal tip electrode 134. This electrode configuration allows for directional or omnidirectional stimulation as may be required for specific applications. The electrodes 134, 136 may be constructed from biocompatible conductive materials such as platinum, platinum-iridium alloy, stainless steel, or other suitable conductive materials.

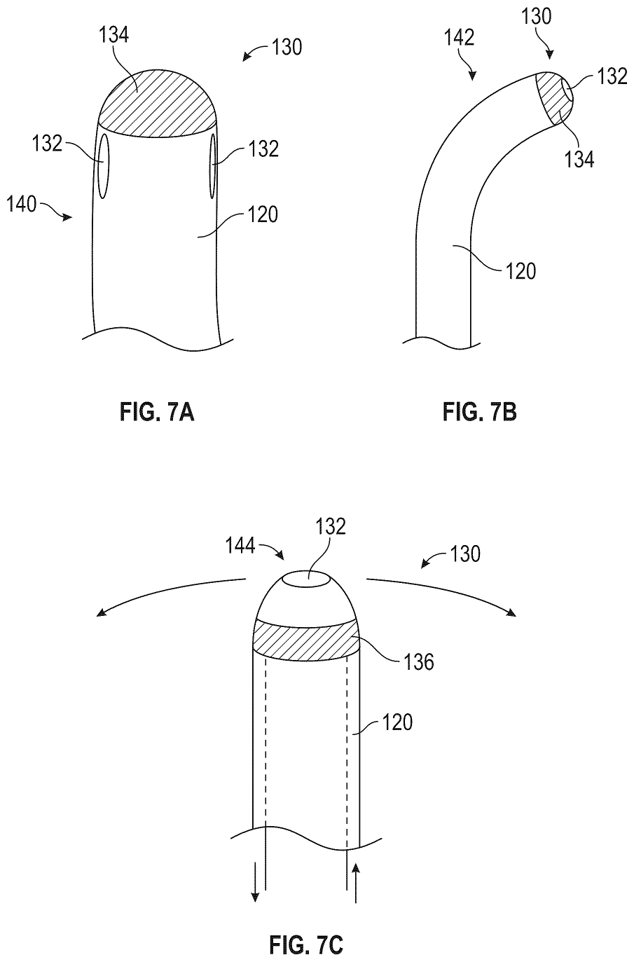

Referring to FIGS. 7A, 7B, and 7C, various distal tip designs may be provided for the catheter 120, each optimized for different anatomical targets and stimulation requirements. In a first embodiment 140, as shown in FIG. 7A, the distal end 130 may include a rounded tip with a distal tip electrode 134 and multiple exit ports 132 arranged circumferentially. In a second embodiment 142, as shown in FIG. 7B, the distal end 130 may include an angled tip to facilitate navigation around anatomical structures. In a third embodiment 144, as shown in FIG. 7C, the distal end 130 may include a steerable tip that can be adjusted by the user to access difficult-to-reach anatomical locations.

Referring back to FIGS. 6A-6B, electrical conductors 146, which may be insulated metal wires, conductive polymer pathways, or other suitable conductive elements, may extend through the catheter wall or through dedicated conduits within the tubular body 122. These electrical conductors 146 connect the electrodes 134, 136 to a connector 148 at the proximal end 128 of the catheter 120. The connector 148 may be configured to interface with the stimulation generator 52 of the neural monitoring system 10, thereby enabling the delivery of electrical stimuli to the nerve via the electrodes 134, 136.

The catheter 120 may include a hub 150 at the proximal end 128 that provides a connection point for both the delivery of therapeutic agents and the electrical connection to the neural monitoring system 10. The hub 150 may include a fluid port 152 in communication with the central lumen 126 and an electrical interface 154 connected to the electrical conductors 146.

In some embodiments, the electrical conductors 146 within the catheter 120 may include shielding to reduce electromagnetic interference. The shielding may be connected to a ground reference at the connector 148. This shielding arrangement may be particularly beneficial when the catheter 120 is used in environments with other electrical equipment, such as fluoroscopy or cautery units, which may generate electromagnetic fields that could potentially interfere with the stimulation or sensing functions.

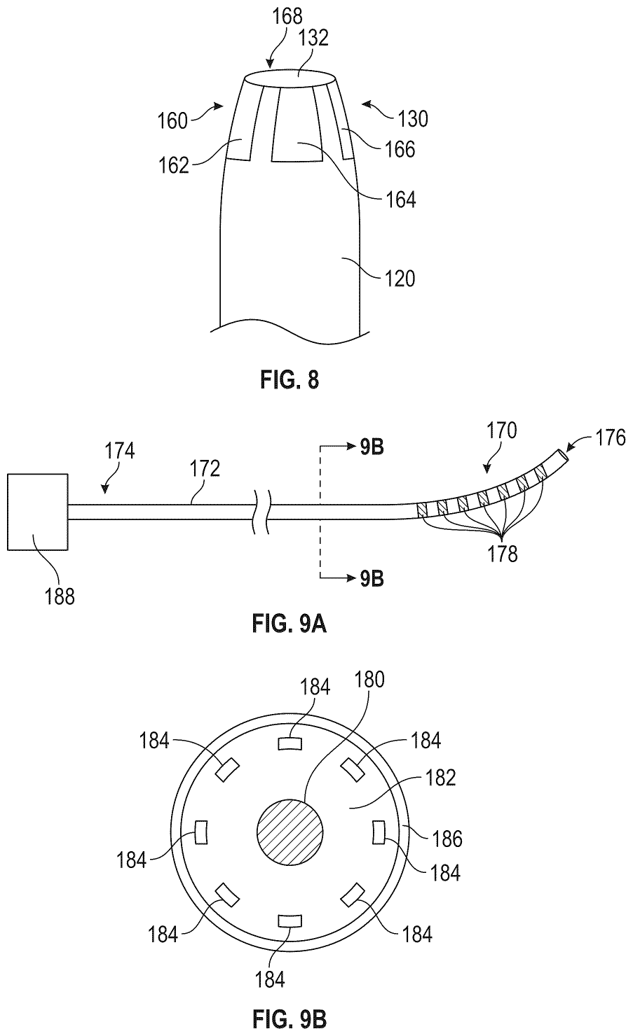

Referring to FIG. 8, for applications requiring precise directional stimulation, in some embodiments, the catheter 120 may incorporate a segmented electrode design 160 with multiple independently controllable electrode segments 162, 164, 166, 168 arranged around the circumference of the distal end 130. This segmented design may allow for selective stimulation in specific directions to target particular nerve structures while minimizing stimulation of surrounding tissues.

### Flexible Multi-Electrode Stimulator

In some embodiments, as an alternative to the stimulated catheter 120 described above, the neural monitoring system 10 may utilize a probe 170 similar to what is typically used in dorsal root ganglion (DRG) stimulator technology. This approach leverages a design familiar to many pain management specialists and offers several advantages for precise nerve localization and stimulation in clinical applications.

Referring to FIGS. 9A-9B, an embodiment of the flexible multi-electrode diagnostic probe 170 is illustrated in detail. As shown, the probe 170 comprises an elongate flexible body 172 with a proximal portion 174 and a distal portion 176. The body 172 may have a length between approximately 30-50 cm and a diameter between approximately 0.8-2.0 mm, designed to be small enough to fit through a 14-gauge needle.

The distal portion 176 includes multiple electrodes 178 arranged in a linear array along the longitudinal axis of the probe. In the illustrated embodiment, eight electrodes 178 are provided, each having a cylindrical configuration with a length of approximately 3 mm and a spacing of approximately 4 mm between adjacent contacts. In other embodiments, the distal portion 176 may include between 4 and 12 electrodes, each having a length between about 1 mm and 5 mm and spacing between about 2 mm and 8 mm between adjacent electrodes. The electrodes 178 may be constructed from biocompatible conductive materials such as platinum, platinum-iridium alloy, gold, or other suitable metals that provide reliable electrical performance and visibility under fluoroscopy, and may be partially embedded within the flexible body 172 to provide a smooth external profile while maintaining appropriate surface exposure for electrical contact with surrounding tissue.

As generally illustrated in FIG. 9B, the flexible body 172 may have a multi-layer design, with an inner core 180 providing structural support, an intermediate layer 182 housing the electrical conductors 184, and an outer jacket 186 providing biocompatibility and appropriate surface properties. The inner core 180 may comprise a flexible polymer such as polyether ether ketone (PEEK) or a flexible metal coil or braid that provides the necessary combination of flexibility for navigation and torsional rigidity for controlled advancement. The intermediate layer 182 may comprise a polymer matrix that electrically insulates the individual conductors 184 from each other while maintaining overall flexibility of the probe. The outer jacket 186 may comprise a biocompatible polymer such as polyurethane or silicone, potentially treated with a hydrophilic coating to reduce friction during insertion.

The electrical conductors 184 extend from each electrode 178 through the length of the flexible body 172 to the connector 188 at the proximal end 174. Each conductor 184 may comprise a thin, flexible wire made of a conductive material such as silver-plated copper, MP35N, or a similar alloy that provides the necessary electrical properties while maintaining flexibility and fatigue resistance. In some embodiments, the conductors 184 may be configured in a helical pattern around the inner core 180 to accommodate flexion of the probe without creating tension or compression in the conductors themselves.

Each electrode 178 may be independently connected to the stimulation generator 52 via separate electrical conductors, enabling various stimulation configurations with electrodes selectively activated as cathodes or anodes for testing in either bipolar or monopolar modes. The connector 188 at the proximal end 174 of the probe 170 includes multiple electrical contacts, each corresponding to different respective electrodes 178, allowing for independent control of stimulation parameters.

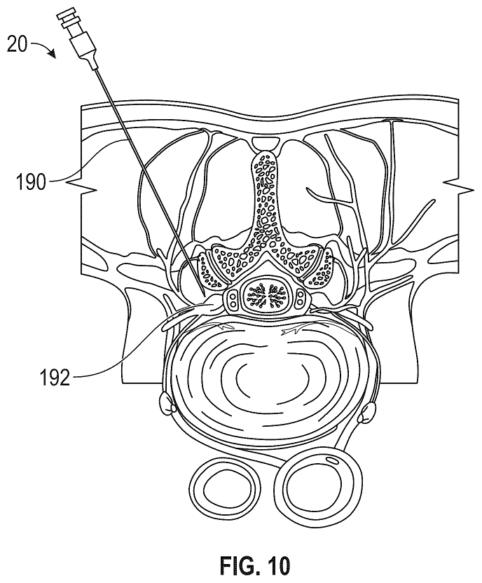

In use, the probe 170 may be inserted through a cannulated introducer needle 190, as shown in FIG. 10, with the distal portion 176 advanced under fluoroscopic guidance to position the electrodes 178 in proximity to the target nerve. The processor 50 may then execute an algorithm to systematically activate each electrode 178 to determine which provides the lowest stimulation threshold, indicating closest proximity to the nerve. This capability to assess multiple electrode positions without physically repositioning the entire probe represents a significant advantage over single-electrode designs, potentially reducing procedure time and improving the precision of nerve localization.

The multi-electrode diagnostic probe 170 may be particularly advantageous for clinical nerve assessment due to its narrow diameter, inherent flexibility, and plurality of electrodes along its length—which may aid in ensuring that at least one is as close as possible to the target nerve. Further, if used in a bi-polar configuration, such a stimulator may further be capable of creating small, focused electrical fields that precisely target specific nerve roots while minimizing stimulation of adjacent structures. This focused stimulation capability may enhance the accuracy of nerve function assessment by reducing the likelihood of activating multiple nerve roots simultaneously, which, in some applications, could confound the interpretation of muscle responses.

For clinical applications, the probe 170 may be inserted and positioned using techniques similar to those employed for temporary trial stimulation in DRG therapy. Such an insertion procedure may involve advancing an epidural needle 190 to the neural foramen under fluoroscopic guidance. Once the needle 190 is properly positioned, the probe 170 may be carefully advanced through the needle 190 and positioned in proximity to the target nerve root 192 where the stimulation procedure may be performed. Following the stimulation, the probe 170 may be removed, and the positioned epidural needle may then be used to administer the fluid therapeutic agent.

In some embodiments, the multi-electrode diagnostic probe 170 may include one or more steering features at its distal portion 176 to facilitate precise positioning near the target nerve. These steering features may include pre-formed curves, deflectable tip mechanisms, or other design elements that assist in navigating the probe 170 to the optimal position for nerve stimulation. The steering capabilities may be particularly valuable when targeting nerve roots in anatomically complex regions or in patients with atypical anatomy due to pathological changes or previous surgical interventions.

Unlike permanent DRG stimulator implants, which are designed for long-term implantation, the probe 170 in the present system is intended for temporary use during clinical assessment procedures. Accordingly, the probe 170 may omit certain features of permanent implants, such as anchoring mechanisms, while incorporating modifications that facilitate efficient insertion, positioning, and removal during a single assessment session.

The processor 50 may execute specialized stimulation protocols stored in memory and optimized for the multi-electrode diagnostic probe 170. These protocols may take advantage of the multiple electrodes 178 to perform advanced stimulation patterns, such as sequential activation of adjacent electrodes, bipolar stimulation between specific electrode pairs, impedance-based tissue classification, or current steering techniques that gradually shift the stimulation field along the electrode array. These patterns may help identify the optimal stimulation location for nerve function assessment or map the functional distribution of a nerve root.

The multi-electrode diagnostic probe 170 may be particularly advantageous for minimizing the separation distance between an electrode and the target nerve. More specifically, the multiple electrodes 178 along the distal portion 176 allow for stimulation at various points along the probe's length without physically repositioning the entire probe. By having the processor 50 execute an algorithm to sequentially activate different electrodes and measure the resulting stimulation thresholds, the system 10 can effectively map the proximity of different sections of the probe to the target nerve. The electrode that yields the lowest stimulation threshold may be identified as being closest to the nerve, and subsequent nerve function measurements can be performed using this optimal electrode, potentially reducing the variability associated with electrode-nerve distance.

### Position Normalization

While the neural monitoring system 10 provides valuable capabilities for nerve function assessment in both surgical and clinical contexts, the clinical application presents a fundamental technical challenge related to the variable distance between the stimulation electrode and the target nerve—particularly if attempting to establish longitudinal trends across different, temporally spaced procedures, possibly performed by different providers. Unlike intraoperative monitoring, where direct visualization and contact with the nerve is possible, clinical procedures must rely on indirect approaches where the stimulation electrode cannot be guaranteed to maintain consistent contact with the target nerve. This distance-dependent relationship introduces significant variability in measurements, as the electrical current required to stimulate a nerve increases approximately 1 mA for each millimeter of separation between the electrode and nerve.

To account for this variability, in some embodiments, the present technology may employ a position normalization framework, executed by the processor 50, that furthers the goal of reliable nerve function assessment across multiple clinical visits, even when direct contact between the stimulation electrode and nerve cannot be guaranteed.

### Fluoroscopic Landmark Registration Method

In accordance with one approach for addressing variable electrode-nerve distance, the neural monitoring system 10 may implement a registration process that relates the stimulator/electrode position to anatomical landmarks visible under fluoroscopic imaging. This registration process may enable estimation of the electrode-nerve distance, which may then be used by the processor 50 to normalize the measured stimulation thresholds.

During a clinical procedure, the physician may use fluoroscopic imaging to guide the placement of the stimulator near the target nerve. The fluoroscopic images may provide visualization of anatomical landmarks (e.g., vertebral bodies, pedicles, foramina) and radiopaque portions of the catheter/electrode.

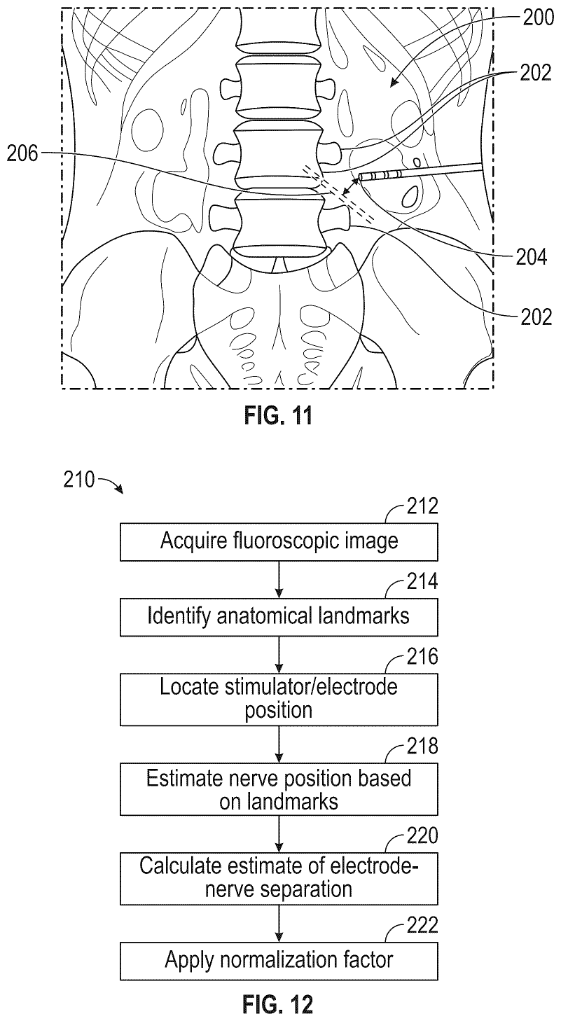

Referring to FIG. 11, the processor 50 may receive fluoroscopic images 200 and execute image processing algorithms stored in memory to identify and track both anatomical landmarks 202 and the stimulator position 204. The anatomical landmarks 202 may serve as reference points for estimating the likely position of the nerve 206. The processor 50 may execute several steps to implement this fluoroscopic landmark registration, including executing an algorithm to identify key anatomical landmarks 202 in the fluoroscopic image 200 (such as pedicles, facet joints, and foramina), locating the radiopaque portions of the stimulator/electrode 204 within the image 200, and calculating the distance between the electrode 204 and the estimated nerve position 206 based on anatomical relationships retrieved from an anatomical model database stored in memory.

The image processing techniques employed may include, but are not limited to, edge detection algorithms to identify vertebral body outlines, feature extraction methods to locate specific anatomical features such as pedicles or facet joints, and template matching to correlate the observed anatomy with standardized anatomical models. In some embodiments, the processor 50 may execute a machine learning-based object recognition algorithm to automatically identify relevant landmarks, potentially increasing the speed and accuracy of the registration process.

Based on the calculated distance, the processor 50, using an established relationship between distance and stimulation threshold retrieved from memory, may apply a correction factor to the measured stimulation threshold. For example, if the calculated distance suggests the electrode is approximately 2 mm from the nerve, the processor 50 may adjust the measured stimulation threshold by a corresponding factor (e.g., approximately 2 mA) to normalize the measurement.

The calculation of correction factors for the stimulation threshold may be based on established or empirically derived relationships between electrode-nerve distance and stimulation requirements. In one embodiment, the correction factor may be calculated by the processor 50 using a linear model, where the relationship between distance and threshold follows the equation:

$$I\_corrected = I\_measured - k \times d$$

where I_corrected is the normalized threshold, I_measured is the measured threshold, d is the estimated electrode-nerve distance, and k is a constant that may be retrieved from memory or a database, having been determined through calibration studies or derived from the literature (i.e., which is believed to approximate a value of 1 for distances less than about 15 mm). In other embodiments, the correction factor may be based on exponential decay functions or a lookup table derived from empirical data stored in a database.

Referring to FIG. 12, a flowchart 210 illustrates an exemplary process for landmark registration and distance normalization executed by the processor 50. The process may begin with the acquisition of a fluoroscopic image at step 212. At step 214, the processor 50 may identify anatomical landmarks within the image. At step 216, the processor 50 may locate the stimulator/electrode position. At step 218, the processor 50 may estimate the nerve position based on the identified landmarks. At step 220, the processor 50 may calculate the electrode-nerve distance. At step 222, the processor 50 may apply a normalization factor to the measured stimulation threshold based on the calculated distance. At step 224, the processor 50 may output the normalized stimulation threshold value for display or storage.

### Position Optimization Technique

Another approach for addressing variable electrode-nerve distance involves a systematic technique for optimizing the stimulator position to achieve the closest possible proximity to the target nerve. This approach may leverage the principle that the minimum stimulation threshold occurs when the electrode is at its closest point to the nerve.

In this approach, the control unit 60 may provide instructions on the display 40 to the physician to systematically adjust the stimulator/electrode position within a confined region near the target nerve while the processor 50 continuously monitors the stimulation threshold. The processor 50 may execute an algorithm to record multiple stimulation thresholds at different positions and identify the minimum threshold, which may correspond to the closest position of the electrode to the nerve.

In some embodiments, the position adjustment process may be guided by specific protocols retrieved from memory to ensure systematic exploration of the target region. For example, the physician may be instructed to move the catheter in a specified pattern, such as a spiral pattern, a grid-like pattern, or along predefined axes, while maintaining visibility under fluoroscopy.

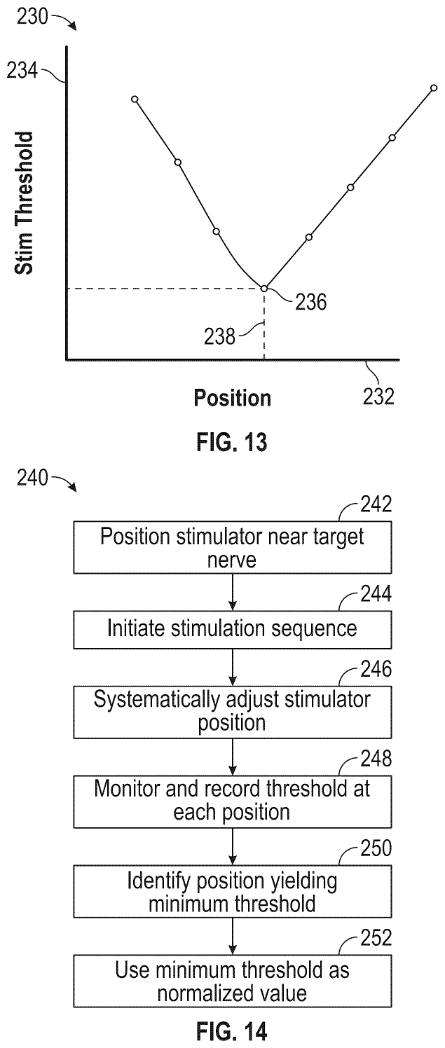

Referring to FIG. 13, a graphical representation 230 illustrates stimulation thresholds recorded by the processor 50 during a catheter position optimization procedure. The horizontal axis 232 represents different catheter positions, while the vertical axis 234 represents the corresponding stimulation thresholds. The graph shows how the stimulation threshold varies with catheter position, with a minimum value 236 occurring at a specific position 238. This minimum value 236 may represent the measurement taken when the electrode is at its closest approach to the nerve.

The processor 50 may continuously execute an algorithm to analyze the stimulus-response relationship during this position optimization process, recording various stimulation thresholds obtained during the targeting process. The processor 50 may then identify the minimum stimulation threshold obtained through this process. In some embodiments, the system may further record the visual fluoroscopy image corresponding to the minimum stimulation threshold, which may then be used in a landmark registration normalization method as described above.

In embodiments where the stimulator includes multiple spaced electrodes along a portion of its longitudinal dimension, the processor 50 may first execute an algorithm to identify the 1-3 electrodes that, when stimulated, yield the lowest stimulation threshold. The processor 50 may then systematically stimulate each electrode to track the minimum stimulation threshold on each electrode during the repositioning/movement and then use the lowest minimum as the final minimum value 236. In doing so, the overall movement required during the targeting may be minimized (or possibly not required at all).

Once the minimum stimulation threshold is identified, this value may be used as a normalized reference point for the procedure, effectively representing the measurement taken at the minimum achievable electrode-nerve distance for that specific anatomical configuration.

For longitudinal tracking, subsequent procedures may employ the same position optimization technique to find the minimum stimulation threshold, allowing for comparisons between sessions that minimize the variability due to electrode placement.

Referring to FIG. 14, a flowchart 240 illustrates an exemplary process for stimulator/electrode position optimization executed by the processor 50. The process may begin with positioning the stimulator/electrode near the target nerve at step 242. At step 244, the processor 50 may initiate a stimulation sequence. At step 246, the physician may systematically adjust the stimulator/electrode position according to a predetermined pattern. At step 248, the processor 50 may monitor and record stimulation thresholds at each position. At step 250, the processor 50 may identify the position yielding the minimum stimulation threshold. At step 252, the processor 50 may utilize the minimum stimulation threshold as the normalized value for assessment and comparison.

### Combined Approach for Enhanced Reliability

In some embodiments, the processor 50 may execute an algorithm that employs a combined approach that leverages both the fluoroscopic landmark registration method and the catheter position optimization technique. This combined approach may provide enhanced reliability through complementary methodologies.

The combined approach may offer several potential advantages. The fluoroscopic registration may provide an initial position reference, while the optimization technique may account for individual anatomical variations and ensure the closest possible position relative to the nerve is achieved.

In some embodiments, the processor 50 may execute an algorithm to compare the minimum stimulation threshold obtained through position optimization with the expected threshold based on the fluoroscopic registration. A significant discrepancy between these values may indicate unusual anatomy or technical issues with the procedure, and the processer 50 may then prompt the physician to reassess the approach.

In some embodiments, the processor 50 may incorporate machine learning algorithms that improve the accuracy of both the landmark registration and position optimization techniques over time. These algorithms may be trained on a dataset of procedures where both techniques were employed, learning to predict optimal catheter positions based on fluoroscopic images and initial stimulation responses. As the system accumulates data from more procedures in its database, the processor 50 can periodically re-train the models to improve predictive accuracy, potentially reducing the time required for position optimization and enhancing the reliability of the normalized measurements.

The processor 50 may further execute confidence estimation procedures for the normalization process. For fluoroscopic registration, confidence metrics may include the number of successfully identified landmarks, the registration error between the observed and expected landmark positions, and the clarity of the fluoroscopic image. For position optimization, confidence metrics may include the depth and definition of the minimum threshold point, the consistency of threshold measurements during the optimization process, and the spatial resolution of the exploration pattern. These confidence metrics may be presented to the healthcare provider alongside the normalized measurements, enabling more informed interpretation of the results.

### Impedance-Based Tissue Differentiation and Threshold Compensation

In some embodiments, the neural monitoring system 10 may implement impedance-based tissue differentiation to enhance the accuracy and reliability of nerve function assessment. This capability addresses significant technical challenges in both surgical and clinical contexts: determining whether the stimulating electrode 26 is in direct contact with the target nerve or separated from it by intervening tissue, such as scar tissue, connective tissue, or other biological structures; and compensating for the effects that different tissue types have on stimulation current flow and nerve activation thresholds. The presence of intervening tissue can significantly alter stimulation threshold measurements in two ways: by creating physical separation distance between the electrode and nerve (with approximately 1 mA increase in threshold per 1 mm of separation), and by modifying current distribution patterns due to the electrical properties of the intervening tissue.

The impedance-based tissue differentiation approach leverages the distinct electrical properties of different biological tissues within the spinal environment. Each tissue type—including neural tissue, scar tissue, muscle, adipose tissue, connective tissue, and cerebrospinal fluid—exhibits characteristic impedance signatures that can be measured and identified across multiple electrical parameters including impedance magnitude, phase angle, and frequency-dependent spectral characteristics. By having the processor 50 execute an algorithm to analyze these impedance signatures, the system 10 can determine the type of tissue at the electrode interface, which may enable the processor 50 to provide an alert to the surgeon and/or to apply appropriate compensation algorithms to adjust stimulation threshold measurements accordingly.

In various embodiments, the system 10 may utilize the impedance-based measurements to operate in one or more distinct modes. In a diagnostic mode, the processor 50 may be configured primarily to identify and report the tissue type at the electrode interface to the healthcare provider via the display 40, thereby providing contextual awareness and alerting the surgeon to reposition the electrode when intervening tissue is detected. In a compensation mode, the processor 50 may automatically execute an algorithm to calculate and display an adjusted stimulation threshold that accounts for both the separation distance and the electrical properties of the identified tissue. In a combined mode, the processor 50 may control the display 40 to show the identified tissue type, estimated separation distance, the raw measured threshold, and the impedance-compensated threshold value simultaneously, offering a comprehensive set of information to the healthcare provider for clinical interpretation. The selection of the operational mode may be user-configurable or determined automatically by the system based on the procedural context.

To implement this functionality, the system 10 may utilize impedance measurement circuitry and a waveform generator, which may be functionally integrated within the system or in communication with the processor 50. The impedance measurement hardware may comprise a voltage controlled current source configured in a multi-electrode measurement arrangement. The current source may deliver controlled alternating current signals within a suitable microampere range across the frequency spectrum, with appropriate current stability and low distortion characteristics. The hardware may include suitable amplification with sufficient common-mode rejection and input impedance characteristics to ensure accurate measurements across varying tissue types.

This hardware may be configured to perform multi-frequency impedance spectroscopy by delivering small-amplitude alternating current signals across a specific frequency range to the electrode 26 and measuring the resulting voltage response. In one embodiment, the frequency range may span from about 200 Hz to about 100 kHz, or within the range of between about 1 kHz and about 50 kHz, which encompasses the β-dispersion region where cell membrane capacitance significantly influences tissue impedance and provides optimal discrimination between different tissue types.

The signal processing unit may implement real-time digital signal processing using appropriate analog-to-digital conversion with suitable sampling rates and resolution for the application. The processor 50 may execute an algorithm to perform frequency domain analysis using appropriate windowing and demodulation techniques to extract impedance magnitude and phase information at multiple frequencies simultaneously.

The impedance measurement may be performed using the same electrodes 26 that are used for stimulation, with the system 10 configured to alternate between (or concurrently output) stimulation and impedance measurement functions in a multiplexed manner. From this, the processor 50 may execute an algorithm to analyze the acquired impedance data to extract key features that characterize the tissue type. Feature extraction may include calculation of impedance magnitude, phase angle, spectral slope derived from frequency response curve fitting, and multi-frequency impedance parameters. The processor 50 compares these extracted features to a reference database of tissue impedance characteristics stored in memory. For example, neural tissue typically exhibits impedance values of approximately 800-4000Ω with a phase angle of 5-10 degrees, while scar tissue may exhibit lower impedance patterns (approximately 150-300Ω) and a reduced phase angle (typically below 5 degrees) compared to healthy neural tissue. High-impedance tissues such as adipose tissue (1300-2000Ω) may indicate significant separation from the target nerve.

The processor 50 may execute machine learning algorithms, such as support vector machines (SVM) or other suitable classification methods, trained on appropriate tissue datasets to distinguish between different tissue types. The training dataset, stored in a database, may comprise samples of various tissue types relevant to spinal procedures, with suitable validation methodologies to ensure classification accuracy. The classification system may achieve sufficient accuracy with appropriate latency for real-time clinical operation.

Once the tissue type and separation are identified, the processor 50 may execute a threshold compensation algorithm that addresses two primary mechanisms affecting nerve stimulation: distance-related threshold increases (using algorithms such as discussed above—approximately 1 mA per 1 mm of separation) and tissue-specific current distribution effects. For example, if the processor 50 detects low-impedance tissue (e.g., muscle, blood) surrounding the nerve, it may determine that additional current is needed to overcome current shunting, where stimulation current dissipates into the conductive tissue rather than activating the target nerve. Conversely, if the processor 50 detects high-impedance tissue (e.g., adipose tissue) between the electrode and nerve, it may determine that the tissue acts as both a physical separator and an electrical barrier that impedes current flow to the nerve.

This compensation may be implemented by the processor 50 using a function such as:

$$I\_adjusted = I\_measured \times f ( Z , P A , S , d\_est )$$

where I_adjusted is the compensated threshold value, I_measured is the raw measured threshold, and f(Z, PA, S, d_est) is a compensation function that depends on impedance magnitude (Z), phase angle (PA), spectral slope (S), and estimated electrode-to-nerve distance (d_est). The compensation function may incorporate impedance-based correction, distance-based correction, and phase angle deviation correction factors. The processor 50 may control the display 40 to show the raw threshold, impedance-compensated threshold, estimated tissue type, and estimated separation distance with appropriate update rates and signal processing for stable clinical visualization.

The processor 50 may incorporate adaptive learning mechanisms with model updating capabilities when classification performance metrics indicate a need for refinement. Confidence assessment may be performed using suitable statistical methods, with appropriate confidence thresholds for clinical decision support. The processor 50 may provide real-time feedback to guide electrode repositioning when intervening tissue is detected with sufficient confidence, thereby improving the safety and efficacy of nerve monitoring procedures.

This impedance-based capability may be particularly valuable in revision surgeries where scar tissue is common, and may be combined with position normalization techniques to further improve the comparability of measurements across different sessions. By identifying the tissue type and separation distance at the electrode-tissue interface and compensating for both distance and tissue-specific effects, the system 10 may provide healthcare providers with more accurate alerts regarding electrode positioning and clinically relevant information about the functional status of neural structures.

### Longitudinal Tracking and Analysis

Longitudinal tracking of nerve function represents a unique capability of the neural monitoring system 10 in clinical applications, enabling the capture and analysis of nerve health parameters across multiple clinical encounters spanning weeks or months of treatment. Unlike intraoperative monitoring, which provides a snapshot of nerve function at a single timepoint, longitudinal tracking reveals the temporal evolution of nerve health, offering insights into disease progression, treatment responses, and optimal timing for surgical intervention. This extended temporal perspective transforms discrete measurements into a continuous narrative of nerve function that can inform clinical decision-making throughout the course of clinical care.

In some embodiments, the processor 50 may execute a longitudinal tracking and analysis framework that addresses the inherent challenges of comparing measurements obtained at different times and under varying conditions. This framework may encompass several potentially interconnected components, including, for example: data normalization algorithms that ensure comparability across sessions despite procedural variations; trend analysis algorithms that identify meaningful patterns in the temporal data; multi-dimensional assessment algorithms that integrate objective nerve function data with subjective patient experiences; and/or specialized visualization tools that transform complex longitudinal data into intuitive, actionable information. These components may enable healthcare providers to detect subtle changes in nerve function over time, distinguish clinically significant trends from random variations, correlate physiological changes with symptomatic presentations, and make data-driven decisions about ongoing management and the potential need for surgical intervention.

### Data Normalization Techniques

In some embodiments, the processor 50 may execute one or more data normalization algorithms stored in memory that aid in normalizing data and comparisons across different assessment sessions and between patients, thus enhancing the clinical utility of the information. While such algorithms may not be strictly necessary, they may aid in improving the confidence or statistical certainty in any given parameter over time.

One approach that may be utilized by the processor 50 is z-score normalization, which transforms raw nerve function measurements into standardized scores. This method may calculate the number of standard deviations a given measurement deviates from the mean of a reference population, where the reference population data is retrieved from a historical patient database. This allows for standardized comparisons across different nerve roots and patients. The processor 50 may also execute min-max scaling algorithms, which bring all parameters to a common scale while preserving the relative differences in the data.

To account for patient-specific variations over time, in some embodiments, the processor 50 may execute an adaptive baseline correction method. This approach maintains a rolling baseline for each patient, continuously updated and stored in a database with each session. New measurements are then normalized against this adaptive baseline, ensuring that the system remains sensitive to clinically significant changes while accounting for natural fluctuations in nerve function.

For longitudinal data analysis, the processor 50 can apply time-series specific normalization techniques. These methods align and normalize measurements across different time points, accounting for variations in the timing of sessions and allowing for more accurate trend analysis.

The processor 50 may also leverage machine learning-based normalization approaches. For instance, autoencoders or other dimensionality reduction techniques can be executed to learn a normalized representation of the data that captures the most important features while minimizing session-to-session variability. This approach allows the system to adapt to complex patterns in the data that may not be apparent through traditional statistical methods.

To address potential variations due to external factors, the processor 50 can incorporate physiological state normalization. This method takes into account measurements of the patient's physiological state, such as heart rate, blood pressure, weight, age, sex, or hydration, and uses these to normalize the nerve function data. This approach may help to account for variations in nerve function measurements that may be due to systemic factors rather than changes in the nerve itself.

Through the application of these advanced normalization techniques, either individually or in combination as appropriate, the nerve function assessment system may ensure that data from different sessions can be meaningfully compared. This capability allows for accurate tracking of nerve function over time and across patients, while accounting for various sources of variability inherent in neurophysiological measurements.

### Trend Analysis Methods

In some embodiments, the system's analysis capabilities may extend beyond individual assessments to include longitudinal trend analysis. By executing an algorithm to track changes in nerve function parameters over time, the processor 50 can identify patterns of improvement, stability, or deterioration. This trend analysis can be particularly valuable in monitoring the progression of chronic pain conditions and assessing the long-term efficacy of various treatment interventions.

The processor 50 may execute various analytical methods to identify clinically significant trends in longitudinal nerve function data retrieved from the database. These methods may include both basic and advanced approaches. Basic trend detection may involve calculating moving averages to smooth out short-term fluctuations and highlight longer-term trends. More sophisticated approaches may include linear and/or non-linear regression analysis to quantify the rate and pattern of change in nerve function parameters over time. For detecting subtle or complex patterns, the processor 50 may execute change-point detection algorithms that identify significant inflection points in the longitudinal data. These algorithms can distinguish between random variations and statistically significant changes in the trajectory of nerve function parameters, potentially signaling disease progression or treatment response. Advanced time series analysis methods such as autoregressive integrated moving average (ARIMA) models may be employed to detect underlying patterns in the data. For extrapolation and future predictions, the processor 50 may incorporate regression models, selecting the best-fit model based on the observed data patterns. Machine learning algorithms, such as recurrent neural networks, may also be executed for more complex pattern recognition and future state prediction, providing healthcare providers with data-driven projections of future nerve function to aid in long-term treatment planning.

The determination of clinically significant thresholds for intervention may be based on statistical analysis of historical data. The processor 50 may retrieve these thresholds from memory, where they may be defined as absolute values (e.g., stimulation threshold exceeding a certain level), relative changes (e.g., percentage increase from baseline), or slope-based criteria (e.g., rate of deterioration exceeding a predetermined value). In some embodiments, these thresholds may be population-based and derived from statistical analysis of outcomes in historical patient cohorts stored in the database. In other embodiments, the thresholds may be individualized based on patient-specific factors such as age, gender, baseline nerve function, and comorbidities.

The longitudinal database structure may organize data in a hierarchical manner, with patient-level data at the highest level, followed by nerve-specific data, and session-specific measurements at the most granular level. This structure may enable efficient querying and analysis across different dimensions, such as tracking a specific nerve root across multiple sessions, comparing different nerve roots within the same session, or aggregating data across multiple patients with similar characteristics. The database may further implement data integrity controls, version tracking, and audit trails to ensure the reliability and traceability of the longitudinal data.

The processor 50 may execute advanced statistical techniques such as mixed-effects modeling to account for both fixed effects (e.g., treatment type, patient demographics) and random effects (e.g., inter-patient variability, measurement noise) when analyzing longitudinal trends. This approach may enhance the system's ability to distinguish between clinically significant changes and random fluctuations, potentially increasing the specificity and sensitivity of trend detection.

To enhance the clinical relevance of the data, the processor 50 may execute an algorithm to integrate the analyzed nerve function parameters with other pertinent clinical information. This may include correlating the objective nerve function data with patient-reported pain scores, functional assessments, and quality of life measures. By presenting this integrated information in an intuitive format, the system aids healthcare providers in developing a comprehensive understanding of each patient's condition.

### Longitudinal Visualization Tools

To facilitate the interpretation of complex longitudinal data, the processor 50 may incorporate specialized visualization tools designed specifically for temporal nerve function data. These visualizations transform raw measurements and analyses into intuitive, actionable information that supports clinical decision-making.

A primary visualization tool may be the multi-nerve tracking graph 100, as illustrated in FIG. 4. This graph, generated by the processor 50 for display, plots nerve function parameters (e.g., stimulation threshold) on the vertical axis 109a against time or clinical visits on the horizontal axis 109b, with separate trend lines for each monitored nerve root. Color-coding may be used to distinguish between different nerves, while shaded or otherwise demarcated regions may indicate normal or target ranges retrieved from a database. A lower region 110 may indicate a range for continued monitoring, while an upper region 112 may indicate a range where surgical intervention might be considered. This visualization allows healthcare providers to quickly identify which nerve roots show persistent abnormalities, which are improving with treatment, and which remain within normal ranges, facilitating targeted treatment planning.

The processor 50 may also provide comparative visualization techniques that juxtapose current measurements with historical data or normative ranges. These comparisons may be presented as side-by-side graphs, overlaid trend lines, or delta analyses highlighting the magnitude and direction of change between time points. Such visualizations help healthcare providers contextualize current findings within the broader trajectory of the patient's condition.

To enhance clinical relevance, the visualization tools may incorporate integration of clinical events and interventions within the temporal display. Markers or vertical lines on the timeline may indicate when specific interventions were performed (e.g., steroid injections, medication changes, physical therapy sessions), allowing healthcare providers to correlate changes in nerve function with specific treatments. This integration facilitates the assessment of treatment efficacy and may reveal delayed or cumulative effects that might otherwise be overlooked.

For supporting treatment decisions, the processor 50 may execute algorithms to implement decision support visualizations that highlight patterns suggestive of specific clinical actions. For example, the visualization may implement color-coded warning indicators when nerve function parameters show deterioration beyond a certain threshold, or recommendation indicators when patterns suggest a particular intervention might be statistically beneficial. As illustrated in FIG. 4, the graph 100 may include regions corresponding to different clinical decision pathways, with the lower region 110 suggesting continued monitoring and the upper region 112 indicating surgical intervention might be considered.

The longitudinal visualization tools may incorporate interactive features that allow healthcare providers to explore the data according to their specific interests. These features may include the ability to zoom in on specific time periods, filter the display to focus on particular nerve roots, toggle between different nerve function parameters, or overlay additional clinical data such as pain scores or medication usage.

In some embodiments, the processor 50 may generate customized reports that combine the most relevant visualizations for a specific clinical context. For surgical planning, for example, the report might emphasize the longitudinal trends of nerve roots at the planned surgical levels, alongside comparisons with contralateral or adjacent nerve roots. For treatment evaluation, the report might focus on before-and-after comparisons surrounding a specific intervention.

Through these specialized visualization tools, the system 10 transforms complex longitudinal nerve function data into clear, interpretable information that supports clinical reasoning and decision-making. By highlighting relevant patterns and trends, these visualizations help healthcare providers extract actionable insights from the wealth of data collected through longitudinal monitoring, potentially improving the precision and effectiveness of care for patients with spinal nerve compression conditions.

### Clinical Guidance Framework

Beyond basic longitudinal tracking capabilities, the neural monitoring system 10 may implement an integrated clinical guidance framework, executed by the processor 50, that transforms objective nerve function data into actionable clinical recommendations. This framework may serve as a comprehensive clinical decision support system that addresses the core questions facing clinicians: how to interpret complex relationships between objective measurements and subjective experiences, which treatment option is most likely to succeed for a specific patient, and when surgical intervention may be indicated. By integrating multiple analytical components into a cohesive guidance system, the neural monitoring system 10 may transition from a passive measurement tool to an active clinical partner that augments clinical judgment with data-driven insights.

The integrated clinical guidance framework may comprise several interconnected components that work in concert to support treatment optimization. First, pain analytics algorithms executed by the processor 50 may correlate objective nerve function data with subjective patient-reported outcomes to contextualize physiological measurements within the patient's experienced symptoms, creating a comprehensive patient profile. Building upon this contextual understanding, predictive analytics algorithms may assess the likelihood of success for various treatment options based on the patient's specific profile, establishing the foundation for evidence-based treatment selection. These predictions may then inform surgical referral timing algorithms, which analyze longitudinal nerve function trajectories alongside predicted outcomes to identify the optimal timing for surgical intervention, potentially reducing both premature and delayed referrals. These integrated components may share a common machine learning infrastructure that continuously refines its analytical models as new outcome data becomes available, enabling increasingly precise and personalized guidance over time. The framework may be designed to augment rather than replace clinical expertise, providing objective, quantitative support for clinical decisions that have traditionally relied primarily on subjective assessment and clinical judgment.

### Integrated Pain Analytics and Patient-Reported Outcomes (PROs)

In some embodiments, the integrated clinical guidance framework may include an integrated pain analytics framework, executed by the processor 50, that provides comprehensive contextual assessment of a patient's condition by correlating objective nerve function data with subjective patient-reported outcomes. This framework may transform raw physiological measurements and subjective reports into clinically meaningful insights that inform both treatment selection and timing decisions. By having the processor 50 execute an algorithm to integrate these complementary data streams, the system 10 may generate a multi-dimensional assessment that captures both the physiological and experiential aspects of the patient's condition, potentially revealing clinically significant patterns that might be missed when analyzing either data type in isolation.

The multi-dimensional pain and PRO profile may include objective nerve function parameters, potentially represented as a composite nerve function index reflecting the overall functional status of the nerve. This objective data may be augmented with multiple categories of subjective information, including pain intensity scores, qualitative pain descriptors, functional impact assessments, medication usage patterns, and temporal pain patterns. In some embodiments, the processor 50 may receive this subjective data from various sources including patient terminals at a clinic, provider input devices, or via its communication circuitry from mobile computing devices operated by the patient at their residence, or fitness/health tracking wearable devices. This received subjective data is then stored in the database, linked to the patient's objective measurements by a unique patient identifier.

The processor 50 may execute correlation algorithms that analyze the relationship between objective nerve function parameters and subjective pain reports across multiple time points. These algorithms may identify clinically significant patterns such as strong correlation between stimulation threshold and pain intensity (suggesting direct nerve compression), discordance between nerve function and reported pain (suggesting central sensitization or psychological factors), or temporal relationships where changes in nerve function precede or follow changes in reported symptoms. In some embodiments, the processor 50 may execute weighted correlation techniques that prioritize certain data types based on their clinical relevance to specific conditions or treatment decisions.

Based on this multi-dimensional analysis, the processor 50 may execute an algorithm to categorize patients into distinct pain profiles that may inform treatment recommendations and surgical timing decisions. Such profiles may include, for example, predominantly neuropathic, mixed neuropathic/nociceptive, or predominantly central. The classification algorithm may incorporate various factors including correlation strength, temporal patterns, medication response, and functional impact, potentially using machine learning techniques to refine classification accuracy based on historical outcomes. In some embodiments, the processor 50 may execute natural language processing algorithms to analyze free-text pain descriptions provided by patients and spatial mapping to correlate pain distribution with specific nerve roots, further refining these pain profiles.

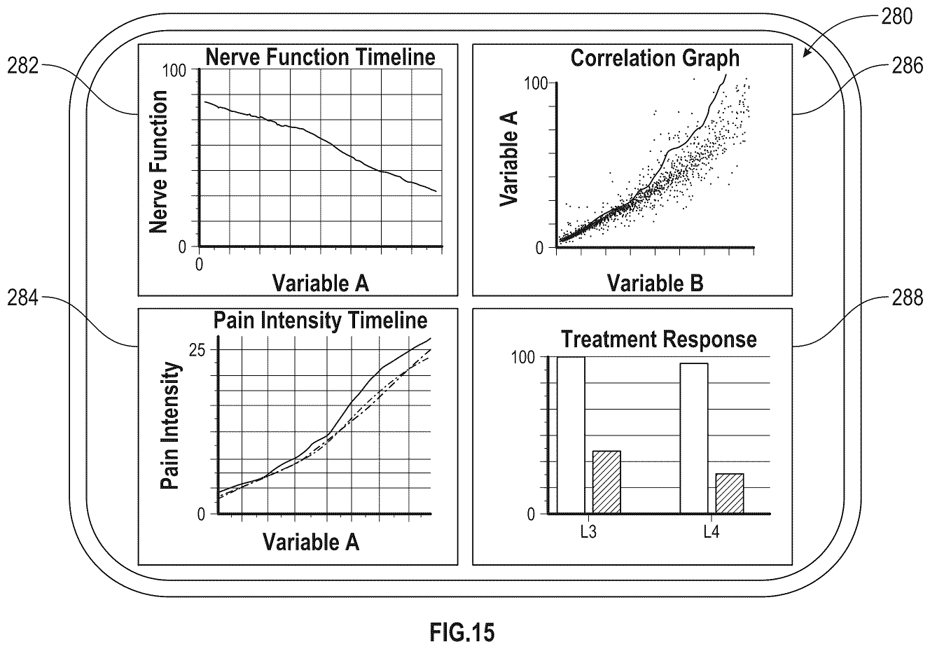

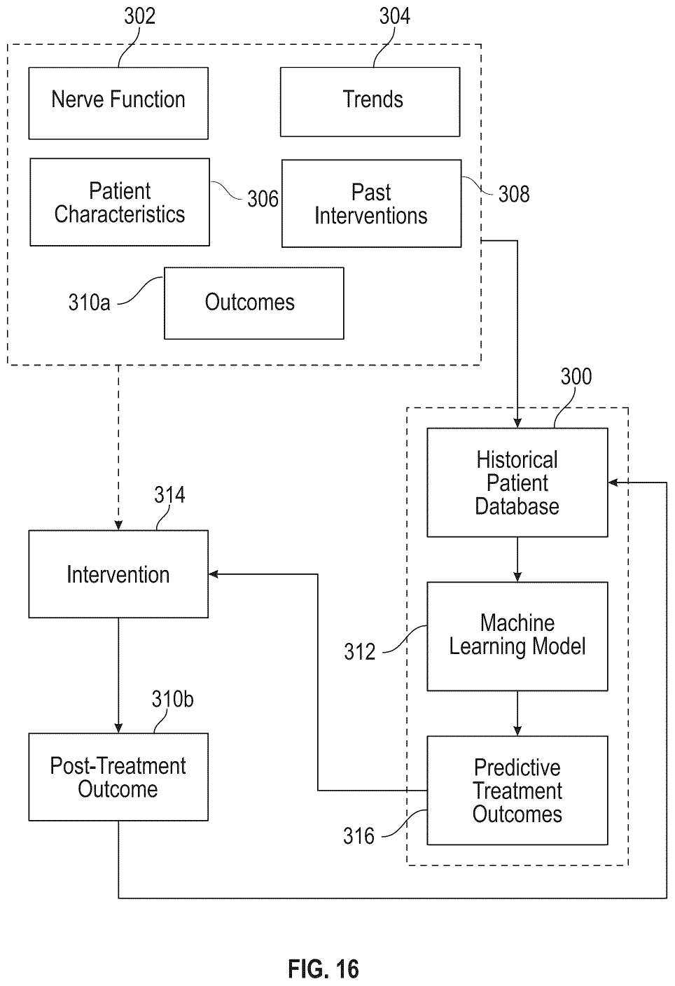

The processor 50 may present this integrated analysis through customizable dashboards that allow healthcare providers to visualize both objective and subjective data streams simultaneously. Referring to FIG. 15, an exemplary dashboard 280 may include a nerve function timeline 282 showing changes in key parameters, a pain intensity timeline 284 showing corresponding changes in reported pain levels, a correlation graph 286 analyzing the relationship between nerve function and pain, and a treatment response section 288 showing how both objective and subjective measures change in response to interventions. In some embodiments, the dashboard may include interactive elements that allow healthcare providers to adjust time scales, highlight specific events, or filter data based on various criteria, potentially enhancing the identification of clinically relevant patterns.

The integrated pain analytics framework may feed its outputs directly into the predictive analytics and surgical timing components of the integrated clinical guidance framework, providing essential context that improves prediction accuracy and recommendation specificity. By executing an algorithm to track changes in the relationship between objective and subjective measures over time, the processor 50 may identify evolving pain mechanisms that warrant treatment adjustments. For example, a diminishing correlation between improving nerve function parameters and persistent pain may indicate the development of central sensitization, which may inform modifications to both treatment selection and surgical timing recommendations. This multi-dimensional approach enables healthcare providers to develop more comprehensive and personalized treatment strategies that address the full spectrum of the patient's pain experience.

### Predictive Analytics for Treatment Outcomes

Building upon the comprehensive patient profiles established by the pain/PRO analytics framework, the processor 50 may leverage machine learning techniques to analyze historical data and generate predictive models for treatment outcomes. By incorporating both objective nerve function parameters and their subjective context, these predictive models may enable healthcare providers to make more informed decisions by estimating the likelihood of success and/or risk of complications for different treatment options.

The processor 50 may execute a supervised learning algorithm trained on comprehensive datasets retrieved from a historical patient database 300. This database is populated over time with data from a plurality of past patients. As generally shown in FIG. 16, for each past patient, the database may contain an entry comprising pre-treatment nerve function parameters 302, longitudinal trends in nerve function parameters 304, patient demographic and clinical characteristics 306, treatment interventions applied 308, and historical post-treatment outcomes 310a. The processor 50, or a remote server in communication with the database, executes a training algorithm on this historical dataset to generate a trained machine learning model 312, which is then stored in memory accessible by the processor 50.

For each potential treatment option or intervention 314 for a current patient (e.g., physical therapy, medication management, targeted injections, surgical intervention), the processor 50 inputs the current patient's specific nerve function profile and characteristics into the trained machine learning model 312 to generate a probability of successful outcome 316.

The processor 50 may present these outcome predictions through a user interface that displays numeric probability of success for each treatment option (e.g., “72% probability of significant improvement with surgical intervention”), confidence intervals for the predictions, comparative visualization of predicted outcomes across treatment options, and similar historical cases and their actual outcomes retrieved from the database. Following the performance of the intervention 312, the processor 50 may feel the post treatment outcome results, such as the pain and PRO assessments, post-intervention clinical evaluations, and other objective measures such as measured nerve parameters (collectively “post-treatment outcomes” 310) back into the historical patient database 300 together with the patient data (302, 304, 306, and/or 308) for that patient, which may be used to further refine the machine learning model 312 for future uses.

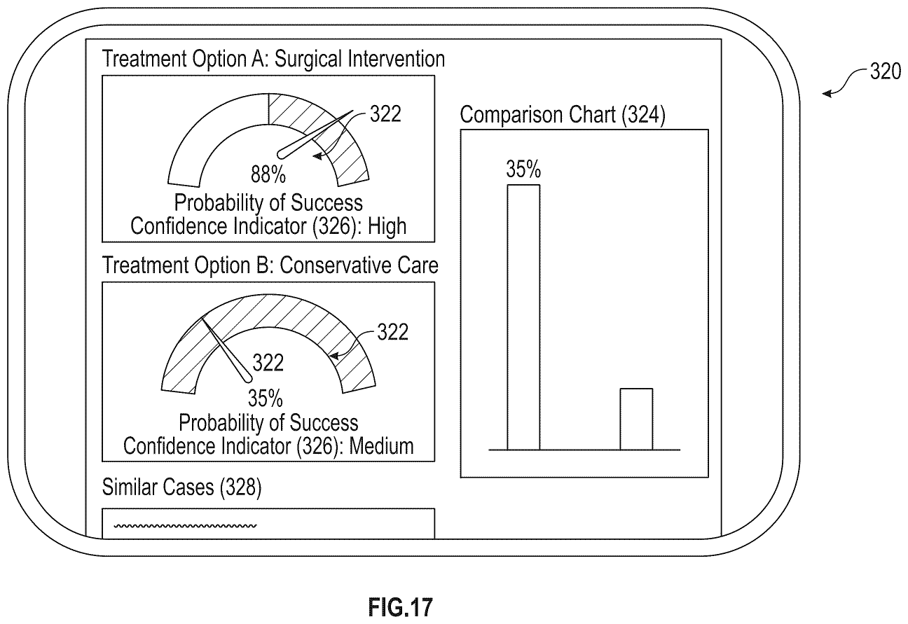

Referring to FIG. 17, an exemplary user interface 320 displays predictive treatment outcomes for a specific patient. The interface 320 may include a probability gauge 322 for each treatment option, showing the likelihood of successful outcome. A comparison chart 324 may allow for side-by-side evaluation of treatment options. A confidence indicator 326 may provide information about the reliability of the predictions. A similar cases section 328 may display anonymized data from patients with similar profiles and their treatment outcomes.

The predictive models may be continuously refined through a feedback loop (as generally illustrated in FIG. 16). As current patients undergo treatments and their outcomes are recorded and added to the historical database, this new information may be used by the processor 50 to periodically re-train and improve the predictive models.

In some embodiments, the processor 50 may execute multiple machine learning algorithms in parallel and compare their predictions. This ensemble approach may provide more robust predictions and highlight areas of uncertainty where different algorithms yield divergent results.

The processor 50 may also calculate and display the expected timeline for improvement with each treatment option. This temporal information may assist in setting appropriate expectations and planning the treatment sequence.

The processor 50 may execute various classes of machine learning algorithms to generate predictive models for treatment outcomes. These algorithms may include, but are not limited to, supervised learning approaches such as random forests, gradient boosting machines, support vector machines, and neural networks. Random forests and gradient boosting machines may be particularly suitable for this application due to their ability to handle mixed data types, robustness to outliers, and inherent feature importance calculation capabilities. Neural networks, including both traditional feed-forward architectures and more advanced recurrent or convolutional architectures, may be employed when sufficient training data is available to leverage their capacity for capturing complex, non-linear relationships.

Feature engineering for the predictive models may involve the processor 50 transforming raw nerve function parameters and contextual data into meaningful inputs for the machine learning algorithms. Time-series features may be derived from the longitudinal nerve function data, including metrics such as slope, volatility, maximum, minimum, and mean values over various time windows. Additional features may be derived from the relationship between different nerve function parameters, such as the ratio between stimulation threshold and saturation threshold or the correlation between threshold changes and response magnitude changes. Patient-specific features may include not only basic demographics but also derived features such as body mass index, duration of symptoms, ratio of imaging findings to functional impairment, and treatment history metrics.

Performance evaluation for the predictive models may utilize multiple complementary metrics to ensure comprehensive assessment. For classification tasks, such as predicting binary treatment success, metrics may include sensitivity, specificity, positive predictive value, negative predictive value, and area under the receiver operating characteristic curve (AUC-ROC). For regression tasks, such as predicting the degree of improvement, metrics may include mean absolute error, root mean squared error, and R-squared. Calibration plots may be used to assess the accuracy of probability estimates, while decision curve analysis may evaluate the clinical utility of the predictions across different threshold probabilities. The system may establish minimum performance thresholds for these metrics based on the clinical context and intended use of the predictions.

To illustrate the predictive analytics capabilities, a machine learning pipeline may be trained on historical patient datasets containing nerve function parameters, patient characteristics, treatment interventions, and outcomes. An exemplary training dataset may include parameters such as patient age, BMI, baseline L5 threshold (mA), L5 threshold slope (mA/visit or mA/week), treatment type, and binary success outcomes.

The processor 50 may execute classification algorithms such as decision tree generators that analyze input features to identify predictive patterns. For example, the algorithm may determine that a baseline L5 threshold>10.0 mA serves as a primary classification criterion, with subsequent nodes based on threshold slope values. The trained model may identify that patients with baseline thresholds of, for example, >10.0 mA and/or threshold slopes>1.2 mA/visit demonstrate significantly higher success probabilities with surgical intervention compared to conservative treatment.

During clinical application, when a patient presents with a baseline L5 threshold of 10.8 mA and demonstrates a threshold slope of 1.6 mA/visit across subsequent visits, the processor 50 may apply the trained model to generate outcome predictions. The processor 50 may calculate, for example, an 88% probability of successful outcome with surgical intervention versus a 35% probability with continued conservative treatment. These predictive outputs may be presented via the user interface 320 as shown in FIG. 17, providing clinicians with quantitative, personalized guidance for treatment selection based on the patient's specific nerve function profile.

Building upon these predictive analytics capabilities, the processor 50 may execute specialized algorithms focused specifically on determining the optimal timing for surgical intervention when surgery is identified as a potentially effective treatment option.

### Surgical Referral Timing Algorithms

When surgical intervention has been identified as a potentially effective treatment option through the predictive analytics described above, the system 10 may further address the critical question of optimal intervention timing. Even when surgery is predicted to be effective based on the comprehensive patient profile that includes both objective and subjective factors, determining the ideal moment for intervention remains a significant clinical challenge. To address this, the longitudinal monitoring capabilities of the neural monitoring system 10 may be leveraged to provide data-driven recommendations regarding when to proceed with surgical intervention, complementing the treatment selection guidance with temporal optimization.

In providing this functionality, the processor 50 may execute algorithms that analyze the trajectory of nerve function parameters over time to identify patterns indicative of a progressive and continuing deterioration of the nerve function or a plateauing response to conservative treatment. These patterns may serve as the basis for generating surgical referral recommendations.

In some embodiments, the processor 50 may execute an algorithm to evaluate several aspects of the longitudinal data to determine optimal surgical referral timing. These aspects may include, for example, the rate of change in nerve function parameters across multiple sessions, the persistence of parameters outside of normative ranges despite conservative treatment, the response magnitude to interventional procedures (e.g., steroid injections), and pattern matching against historical cases with known outcomes retrieved from the database.

The processor 50 may execute an algorithm to classify the longitudinal trends into several categories that may inform the statistical referral recommendation. These categories may include, for example, progressive deterioration, plateau after initial improvement, non-response to conservative treatment, and fluctuating function. Based on this classification and analysis of historical outcome data, the processor 50 may generate a statistically based surgical referral recommendation, which may include an associated confidence level. In some embodiments, this recommendation may include a suggested timeline for referral (e.g., immediate, within 3 months, after additional conservative treatment).

Further, in some embodiments, the processor 50 may execute an algorithm to inform surgical referral recommendations. Such an algorithm may begin with an initial assessment of the patient's nerve function parameters. Based on these parameters, the processor 50 may recommend continued monitoring, conservative treatment, or surgical consultation. Subsequent measurements and trend analysis may lead to refined recommendations through various branches of the decision tree. Following each intervention or period of continued observation, the processor 50 may be operative to reassess and compare more temporally recent measurements against prior measurements to further inform next steps.

In some embodiments, the processor 50 may incorporate patient-specific factors into the surgical referral algorithm. These factors may include, for example, age, gender, body mass index (BMI), comorbidities, duration of symptoms, and previous treatments. By considering these factors alongside the trending nerve function parameters, the processor 50 may generate more personalized recommendations.

The surgical referral algorithms may be continuously refined based on outcome data. As patients undergo different treatments and their outcomes are recorded in the database, this information may be incorporated into the algorithms to improve their predictive accuracy.

In some embodiments, the surgical referral timing algorithms may analyze longitudinal nerve function trajectories to generate data-driven recommendations. For example, the processor 50 may track a patient's L4 nerve root stimulation threshold across multiple clinical visits. At an initial assessment, the processor 50 may determine a baseline threshold of 8.2 mA. Following therapeutic intervention, subsequent measurements may show improvement to 5.1 mA at two weeks, followed by regression to 7.4 mA at six weeks, and further deterioration to 9.8 mA at twelve weeks.

The processor 50 may execute temporal analysis algorithms that perform linear regression analysis on the post-intervention measurements (5.1, 7.4, 9.8 mA), calculating a positive slope of approximately 1.57 mA per visit (or an average of about 0.47 mA/week). When this calculated rate of deterioration exceeds a pre-defined progression alert threshold stored in memory (e.g., >1.0 mA per visit or >0.4 mA per week), the processor 50 may control the display 40 to present a surgical consultation recommendation, as illustrated in the upper region 112 of FIG. 4. This objective, algorithmic approach provides clinicians with quantitative evidence supporting surgical referral decisions based on documented functional deterioration despite conservative treatment.

### Machine Learning Pipeline

Serving as the technical foundation for all components of the integrated clinical guidance framework, the neural monitoring system 10 may implement a comprehensive data integration and machine learning pipeline. This shared infrastructure may power the integrated pain analytics, predictive analytics, and surgical timing algorithms described above, enabling their integration into a cohesive clinical guidance system while supporting continuous improvement of each capability over time.

In one embodiment, the data integration pipeline may begin with data collection from multiple sources, including nerve function measurements, patient-reported outcomes, treatment records, and electronic health record data. The collected data may undergo preprocessing by the processor 50, including cleaning, normalization, and feature extraction. The preprocessed data may then be stored in a structured database.

The machine learning pipeline may be executed by the processor 50, which accesses the structured database and implements multiple algorithms for different analytical tasks. These algorithms may include, for example, classification algorithms for categorizing patients into distinct profiles, regression algorithms for predicting quantitative outcomes, clustering algorithms for identifying natural groupings in the data, and time series analysis algorithms for analyzing longitudinal trends.

The outputs from these algorithms may be integrated and validated before being presented through the user interface. The validation process may include comparison with known outcomes, cross-validation techniques, and expert review.

As new data is collected, it may be incorporated into the database, and the machine learning models may be retrained or fine-tuned by the processor 50. This continuous learning process may enable the system to adapt to new patterns and improve its predictive accuracy over time.

In some embodiments, the system 10 may implement federated learning techniques that allow the models to be improved using data from multiple clinical sites while maintaining patient privacy and data security. This approach may enable faster accumulation of knowledge and more robust predictive models.

The processor 50 may also execute interpretable machine learning models that provide explanations for their predictions. These explanations may include the key factors contributing to a specific prediction and the relative importance of different variables, enhancing transparency and trust in the system's recommendations.

### Wearable Device Integration

In some embodiments, the neural monitoring system 10 may extend its assessment capabilities beyond the clinical setting through integration with commercially available wearable devices and mobile applications. Such an integration bridges the gap between episodic clinical measurements and continuous real-world functioning, providing insights into how nerve compression affects patients' daily activities and how treatments translate into functional improvements. By having the processor 50 retrieve and analyze objective activity data collected between clinical visits, the system 10 enriches its longitudinal perspective with contextual information that may be more representative of the patient's actual quality of life and functional status than periodic clinical assessments alone.

Wearable device integration represents a non-invasive approach to extending the temporal and contextual scope of nerve function monitoring. Through secure communication interfaces with consumer health platforms, the processor 50 can access activity metrics such as step counts, intensity levels, and sleep patterns that may correlate with nerve function status. These metrics not only provide additional objective evidence of treatment efficacy but may also reveal functional impacts of nerve compression that are not apparent during clinical visits. The analysis of these activity patterns in conjunction with nerve function parameters may enhance treatment evaluation, guide intervention timing, and provide patients with personalized feedback to support their recovery process.

The system 10 may implement a comprehensive technical architecture for integrating nerve function data with patient-reported outcomes and wearable device data. This architecture may include a central data repository that serves as the primary storage location for all patient-related data, interface layers that enable communication with external data sources, data processing pipelines executed by the processor 50 that transform and normalize incoming data, and presentation layers that generate integrated visualizations and analyses.

The integration with wearable devices may utilize standard APIs provided by device manufacturers and health data aggregation platforms. In one embodiment, the processor 50, via its communication circuitry 58, may establish connections with platforms such as Apple HealthKit, Google Fit, or Fitbit API, which serve as intermediaries that aggregate data from multiple consumer devices. This approach simplifies the integration process by reducing the number of direct device connections required while expanding the range of supported devices. Communication with these platforms may be implemented using RESTful APIs, GraphQL, or similar standardized approaches, with authentication handled through OAuth 2.0 or similar protocols to ensure secure access to patient data.

The processor 50 may execute data normalization algorithms to address the heterogeneity of data collected from different sources. For wearable device data, normalization may involve converting step counts, activity minutes, or sleep measurements to standardized units and formats, potentially adjusting for differences in device sensitivity or measurement protocols. For patient-reported outcomes, normalization may involve converting responses from different assessment instruments (e.g., Visual Analog Scale, Numerical Rating Scale) to comparable scales, potentially using established conversion factors from the literature. Nerve function parameters may be normalized as described in previous sections to account for procedural variations. These normalized data streams may then be aligned temporally to enable meaningful comparison and correlation analysis.

The correlation analysis between nerve function, patient-reported outcomes, and activity data may employ various statistical approaches executed by the processor 50. Basic approaches may include Pearson or Spearman correlation coefficients to quantify the strength and direction of relationships between different measures. More sophisticated approaches may include mixed-effects models that account for the hierarchical structure of the data and control for potential confounding factors, or time-series analysis methods that identify temporal relationships, such as whether changes in nerve function parameters precede changes in activity levels. Pattern recognition algorithms may be employed to identify characteristic signatures in the integrated data that may have prognostic or diagnostic value.

The system 10 may implement comprehensive security and privacy measures to protect sensitive patient data. These measures may include end-to-end encryption for data transmission, role-based access controls for data access, audit logging for all data-related activities, and data minimization principles to ensure that only necessary information is collected and stored. The system may be designed to comply with relevant regulatory requirements, such as the Health Insurance Portability and Accountability Act (HIPAA) in the United States or the General Data Protection Regulation (GDPR) in Europe, implementing appropriate technical and organizational controls to safeguard patient data throughout its lifecycle.

The correlation between objective nerve function improvements and real-world activity changes may be demonstrated through a wearable device integration framework. For example, following therapeutic intervention, the processor 50 may measure a significant improvement in L4 stimulation threshold from a pre-intervention baseline of 10.5 mA (e.g., measured in week 0) to a post-intervention value of 5.5 mA (e.g., measured at week 4), providing objective evidence of physiological improvement.

To assess functional translation of this improvement, the processor 50 may establish secure communication with health data platforms to retrieve daily step count data from a patient's wearable device. The processor 50 may execute correlation analysis algorithms that calculate 7-day trailing averages to smooth daily variability, revealing an increase from pre-intervention average of 2,100 steps/day to post-intervention average of 4,800 steps/day.

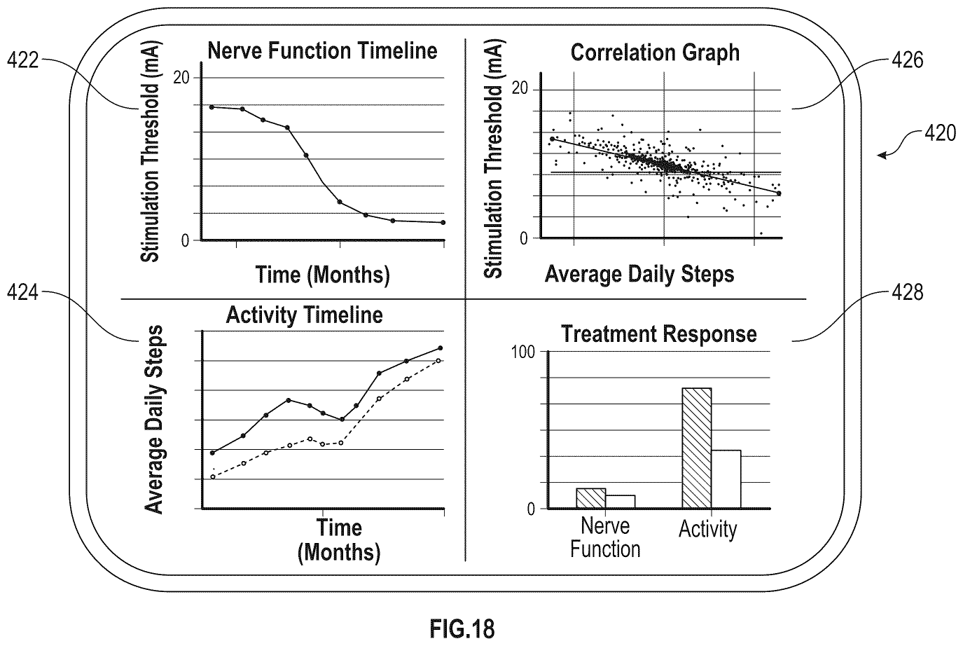

The processor 50 may generate integrated visualizations via the display 40, presenting synchronized timelines showing the sharp decrease in L4 stimulation threshold alongside the corresponding increase in average daily activity, as illustrated in the dashboard of FIG. 18. This correlation analysis provides clinicians with objective evidence that physiological improvements measured during clinical visits translate into meaningful functional improvements in patients' daily activities, thereby validating treatment efficacy through multiple independent metrics.

### Activity Data Collection

In some embodiments, the processor 50 may be configured to receive and process one or more activity-based parameters that are recorded and transmitted to the processor 50 from a personal device of the subject 14. Such activity-based parameters may include, but are not limited to, steps taken, distance traveled, floors climbed, calories burned, active minutes, and intensity levels as detected by connected wearable devices or mobile applications. The processor 50 may also receive and process other physiological parameters that may correlate with nerve function and pain status, such as sleep duration, sleep quality, heart rate patterns, and heart rate variability.

In some embodiments, the system 10 may implement a data integration framework that securely receives activity data from various sources. The framework may include an application programming interface (API), managed by the processor 50, that communicates with health data platforms on the subject's personal device. These health data platforms may aggregate data from multiple third-party wearable devices and applications, acting as an intermediary that simplifies data collection while maintaining privacy and security. The data integration framework may implement appropriate authentication and encryption protocols to ensure secure data transmission and compliance with relevant privacy regulations.

The processor 50 may execute specialized algorithms to analyze the activity data in conjunction with nerve function parameters. These algorithms may identify correlations between changes in nerve function and changes in daily activity patterns, potentially revealing the functional impact of nerve compression or the effectiveness of therapeutic interventions. For example, an increase in average daily steps or active minutes following a therapeutic procedure may provide objective evidence of improvement that complements the nerve function measurements obtained during clinical visits.

The processor 50 may control the display 40 to show activity trends alongside nerve function parameters, providing a comprehensive view of the subject's status over time. This integrated visualization may help clinicians identify discrepancies between objective measurements and subjective reports, such as cases where improved nerve function does not correlate with increased activity or where activity increases despite stable nerve function parameters. Such insights may inform adjustments to treatment plans or help identify psychosocial factors that may be influencing treatment outcomes.

### Real-World Function Assessment

In some embodiments, the processor 50 may execute machine learning algorithms to analyze the relationship between activity patterns and nerve function parameters across multiple subjects. These algorithms may identify characteristic activity signatures associated with different types of nerve compression or predict treatment outcomes based on early changes in activity following intervention. The processor 50 may use these insights to generate personalized activity targets or recommendations that may help optimize recovery and functional improvement.

For subjects with similar nerve function parameters, variations in activity levels may help clinicians distinguish between those who might benefit from continued conservative treatment and those who might require surgical intervention. For example, subjects whose activity levels remain persistently low despite therapeutic interventions may represent candidates for earlier surgical evaluation, while those showing progressive activity improvements might reasonably continue with conservative management.

The processor 50 may further incorporate contextual information to enhance the interpretation of activity data. This may include weather data that might affect outdoor activity, calendar information that might explain changes in routine, or self-reported contextual factors such as pain medication usage. By accounting for these contextual factors, the processor 50 may generate more accurate assessments of the relationship between nerve function and real-world activity.

FIG. 18 illustrates an exemplary dashboard 420 generated by the processor 50 showing the integration of activity data with nerve function parameters. The dashboard 420 may include a nerve function timeline 422 showing changes in key parameters, an activity timeline 424 showing corresponding changes in daily step count or other activity metrics, a correlation graph 426 analyzing the relationship between nerve function and activity, and a treatment response section 428 showing how both objective measures and activity levels change in response to interventions.

By integrating commercially available wearable device data, the system 10 transforms sporadic clinical measurements into a continuous monitoring system that captures the real-world functional impact of nerve compression and treatment. This approach provides clinicians with objective evidence of treatment efficacy between visits and may help identify the optimal timing for surgical intervention based on functional outcomes rather than subjective reports alone.

### Clinical Applications

The neural monitoring system 10 described herein may encompass a range of clinical applications spanning the continuum of care for patients with spinal nerve compression conditions. These applications leverage the system's capabilities for objective nerve function assessment, longitudinal monitoring, and data-driven analysis to address key challenges in diagnosis, treatment planning, and surgical decision-making. By providing quantitative evidence of nerve function status and changes over time, the system 10 complements traditional clinical assessment methods and imaging studies, potentially enhancing both the precision and effectiveness of care throughout the patient journey.

The clinical applications of the system 10 address several persistent challenges in the management of spinal nerve compression conditions. In the diagnostic phase, the system helps identify specific compressed nerve roots and distinguish between various etiologies of neuropathic pain with greater precision than is possible with subjective assessment and imaging alone. For treatment planning and guidance, the system provides real-time feedback during interventional procedures and data-driven recommendations for treatment selection. Through longitudinal monitoring, the system enables objective tracking of disease progression and treatment response over time. For surgical decision-making, the system supports evidence-based referral thresholds and personalized outcome predictions. Throughout this spectrum of applications, the system integrates seamlessly with existing clinical workflows, enhancing rather than disrupting established practices.

### Diagnostic Applications

The nerve function assessment system described herein offers a wide range of clinical applications in pain management, particularly in the context of spinal nerve compression syndromes. One primary application lies in its diagnostic utility. By providing objective, quantifiable data on nerve function, the system 10 can assist healthcare providers in identifying specific compressed nerve roots with a high degree of precision. This capability addresses a significant challenge in current pain management practices, where localizing the exact source of pain can be difficult based solely on clinical examination and imaging studies. Furthermore, the system 10 and use of nerve function parameters may provide clinicians with the ability to differentiate between various etiologies of nerve dysfunction, such as in distinguishing compressive lesions from other causes of neuropathic pain, such as inflammatory or metabolic conditions.

In the diagnostic phase, the system 10 works in concert with traditional assessment methods such as physical examination, medical history review, and imaging studies. The objective nerve function data provided by the system 10 can corroborate or refine clinical impressions, potentially increasing diagnostic accuracy and reducing the need for repetitive or invasive diagnostic procedures. For instance, in cases where imaging studies reveal multiple levels of potential nerve compression, the nerve function assessment can help identify which levels are functionally compromised, guiding more targeted treatment approaches.

The system 10 may be particularly valuable in cases where subjective pain reports do not align with imaging findings. For example, a patient may present with pain patterns suggestive of L5 nerve root compression, while MRI shows compression at multiple levels including L4, L5, and S1. By assessing the functional status of each nerve root, the system 10 can help determine which compression is physiologically significant and likely contributing to the patient's symptoms. This precision in identifying functionally compromised nerve roots may reduce diagnostic uncertainty and potentially decrease the number of unnecessary interventions targeting anatomical findings that may not be clinically relevant.

Beyond identifying the location of nerve compression, the system 10 enables differentiation between various etiologies of neuropathic pain. Different patterns of nerve function parameters may be associated with distinct underlying causes. For example, nerve compression typically presents with elevated stimulation thresholds and reduced maximal response magnitudes, while inflammatory conditions may show different patterns. By having the processor 50 analyze these patterns, healthcare providers can make more informed decisions about appropriate treatment approaches, potentially improving outcomes and reducing the trial-and-error aspect of pain management.

The enhanced diagnostic precision offered by the system 10 may lead to a reduction in unnecessary procedures. By providing clear, objective evidence of which nerve roots are functionally compromised, the system 10 helps healthcare providers focus interventions on the most relevant targets. This targeted approach may reduce the incidence of “diagnostic” procedures that aim to identify the pain source through trial injections at multiple levels. Instead, healthcare providers can proceed more directly to therapeutic interventions at the levels identified as functionally compromised, potentially reducing the overall number of procedures and associated risks.

### Treatment Planning and Guidance

In the realm of treatment planning and guidance, the nerve function assessment system 10 serves as a valuable tool for optimizing interventional procedures. For instance, when performing nerve block injections, healthcare providers can use the system 10 to precisely locate the target nerve and guide the placement of the injection. The real-time feedback provided by the system 10 during these procedures can help ensure that the therapeutic agent is delivered to the most appropriate location (e.g., only the compressed and/or functionally impaired nerves), potentially enhancing the efficacy of the treatment. Additionally, the objective data provided by the system 10 can inform the selection of other treatment modalities, such as physical therapy regimens or medication management strategies, by offering insights into the specific functional deficits associated with the nerve compression.

During interventional procedures, the system 10 can be utilized to enhance precision and efficacy through procedure optimization. The stimulated catheter 120 or probe 170, combined with position normalization techniques, allows for more accurate targeting of specific nerve roots. By monitoring stimulation thresholds during catheter advancement and positioning, healthcare providers can identify the optimal location for therapeutic agent delivery. This precision targeting may improve the efficacy of interventional procedures such as epidural steroid injections or selective nerve root blocks by ensuring that the therapeutic agent reaches the nerve root that is functionally compromised.

The system 10 may also guide therapeutic targeting decisions by providing information about which nerve roots would benefit most from intervention. In cases where multiple nerve roots show compression on imaging, the processor 50 can help prioritize treatment based on the degree of functional impairment. Nerve roots showing more significant functional abnormalities may be targeted first, potentially leading to more efficient symptom resolution. This prioritization may be particularly valuable in planning a series of interventions where treating all potential pain generators simultaneously is not feasible or advisable.

Beyond guiding individual procedures, the system 10 provides treatment selection guidance at a broader level. By having the processor 50 correlate nerve function parameters with responses to various treatment modalities, the system 10 can generate recommendations about which treatment approaches may be most effective for a specific patient's condition. For example, patients with certain patterns of nerve function impairment may respond better to particular medication classes, specific physical therapy protocols, or certain types of interventional procedures. These data-driven recommendations may help healthcare providers select the most appropriate treatment pathway from the outset, potentially reducing the trial-and-error approach that is common in pain management.

The integration of the system 10 with interventional workflows represents a significant enhancement to current practice. The stimulated catheter 120 or probe 170 allows for nerve function assessment to be performed simultaneously with therapeutic procedures, without requiring separate insertions or additional procedural time. This integration may increase the adoption of objective nerve function assessment in routine clinical practice by minimizing disruption to established workflows. Furthermore, the data collected during these integrated procedures contributes to the system's learning capabilities, continuously refining its algorithms and recommendations based on observed outcomes.

### Longitudinal Monitoring Applications

The system's capability for longitudinal monitoring of nerve function represents another potentially valuable clinical application. By having the processor 50 track changes in nerve function parameters over time, healthcare providers can objectively assess the progression of a patient's condition. This feature is particularly valuable in managing chronic pain conditions, where subtle changes in nerve function may precede clinical deterioration. The ability to detect these changes early can allow for timely intervention, potentially preventing further nerve damage and improving long-term outcomes. Moreover, this longitudinal data can be used to evaluate the effectiveness of ongoing treatments, providing an objective basis for adjusting management strategies as needed.

The processor 50 can simultaneously monitor and track multiple nerve roots over time, as illustrated in FIG. 4. This multi-nerve tracking capability allows healthcare providers to compare the functional status of different nerve roots across multiple pre-operative pain injection visits. By visualizing the nerve response threshold for different nerve roots (e.g., L4 left 102, L4 right 104, L5 left 106, and L5 right 108) over time, healthcare providers can identify patterns that may inform clinical decision-making regarding the necessity and focus of surgical interventions.

This visualization showcases patterns such as a consistently elevated response threshold for the L4 left nerve root 102 across all visits, indicating persistent compression and functional impairment throughout the duration of tracking. This pattern suggests it may be the primary source of the patient's symptoms and a potential focus for decompression surgery. In contrast, the L4 right nerve root 104 shows a progressive increase in response threshold over the course of the visits, indicating a worsening condition and potentially representing a growing contributor to the patient's symptoms. Such patterns could inform surgical planning, suggesting that both nerve roots may require attention during intervention. Meanwhile, the L5 left and L5 right nerve roots 106, 108 demonstrate relatively stable and low response thresholds, remaining within or close to the normal/healthy range throughout the monitoring period, suggesting these nerve roots are likely not significant contributors to the patient's condition.

Progression monitoring represents a key aspect of the system's longitudinal capabilities. By having the processor 50 track nerve function parameters over time, healthcare providers can identify trends indicative of disease progression, stabilization, or improvement. This objective tracking provides insights into the natural history of the patient's condition and may help identify cases where nerve compression is progressively worsening despite conservative management. Early identification of such progression may prompt more aggressive intervention before significant or irreversible nerve damage occurs. Conversely, documentation of stable nerve function over time may provide reassurance that conservative management remains appropriate.

The system 10 enables objective assessment of treatment efficacy through comparison of pre-treatment and post-treatment nerve function parameters. After interventions such as epidural steroid injections, nerve blocks, or medication adjustments, changes in nerve function parameters can provide quantitative evidence of physiological improvement, independent of subjective pain relief. This objective assessment helps healthcare providers distinguish between interventions that directly address the underlying nerve dysfunction and those that may provide symptomatic relief through other mechanisms. Such distinctions may inform the long-term treatment strategy, potentially focusing on interventions that show evidence of improving nerve function rather than just masking symptoms.

The integration of longitudinal nerve function data with other clinical information supports decision-making regarding treatment adjustments. For example, if nerve function parameters show improvement following an intervention while pain persists, this discordance may suggest additional pain mechanisms that require different treatment approaches. Conversely, if nerve function parameters remain abnormal despite multiple interventions, this persistence may indicate the need to consider alternative treatments or surgical evaluation. By providing objective data on the physiological effects of treatments over time, the system 10 helps healthcare providers make more informed decisions about when to continue with the current treatment approach, when to modify it, and when to consider more invasive options.

### Surgical Decision Support

In the context of surgical decision-making, the nerve function assessment system 10 can play a pivotal role in informing the timing and necessity of surgical interventions. By providing objective measures of nerve function, the system 10 can help establish evidence-based thresholds for surgical referral. This capability addresses a significant challenge in current practice, where the decision to proceed with surgery is often based on subjective criteria and clinical judgment alone. The system's data can complement clinical assessment and imaging findings, offering a more comprehensive picture of the patient's condition and potentially reducing the incidence of unnecessary surgeries or delays in necessary surgical intervention.

The processor 50 can help establish evidence-based thresholds for surgical referral through analysis of historical data correlating nerve function parameters with surgical outcomes. By identifying patterns in nerve function that are associated with successful surgical results, the processor 50 can generate quantitative thresholds that may indicate when a patient is likely to benefit from surgical intervention. These thresholds may include absolute values of nerve function parameters (e.g., stimulation threshold above a certain level), patterns of change over time (e.g., progressive deterioration despite conservative treatment), or combinations of multiple parameters and clinical factors. By basing these thresholds on actual outcome data rather than subjective assessment alone, the system 10 provides a more objective foundation for surgical decision-making.

Risk-benefit assessment tools executed by the processor 50 can help healthcare providers and patients weigh the potential advantages and disadvantages of surgical intervention. By analyzing the patient's specific nerve function profile alongside demographic and clinical characteristics, the processor 50 can generate personalized estimates of both the likelihood of surgical success and the potential risks of complications. This balanced presentation of potential outcomes supports shared decision-making between healthcare providers and patients, potentially leading to more appropriate surgical referrals and better alignment of expectations with likely outcomes.

For patients proceeding to surgery, the system 10 provides valuable surgical planning support. The longitudinal data collected through clinical monitoring can inform decisions about which nerve roots should be targeted for decompression, the extent of decompression required, and the most appropriate surgical approach. By identifying which nerve roots show the most significant functional impairment and tracking how this impairment has evolved over time, the system 10 helps surgeons focus their intervention on the most clinically relevant targets. This focused approach may reduce unnecessary surgical manipulation of non-contributing nerve roots, potentially decreasing operative time and surgical morbidity.

The predictive analytics capabilities of the system 10 extend to surgical outcome prediction, providing estimates of the likelihood of symptomatic improvement following surgery. These predictions, based on the analysis of nerve function parameters and other patient characteristics, can help set appropriate expectations for both healthcare providers and patients. For example, the processor 50 might identify that a patient with a specific nerve function profile has an 85% probability of significant pain relief following decompression surgery. This type of quantitative prediction may be particularly valuable when counseling patients about surgical options and helping them make informed decisions about whether to proceed with surgery.

### Integration With Clinical Workflows

The nerve function assessment system 10 is designed to seamlessly integrate with and complement existing pain management protocols, enhancing the overall quality of care without disrupting established clinical workflows. This integration is achieved through consideration of current diagnostic and treatment modalities, with the system 10 serving as an additional layer of objective information to support clinical decision-making.

In some embodiments, the processor 50 may be configured to integrate with electronic health record (EHR) systems to facilitate data sharing and documentation. This integration may involve bidirectional communication, with the processor 50 receiving relevant patient information from the EHR and sending nerve function assessment data and analyses back to the EHR for incorporation into the patient's medical record. This seamless data exchange reduces duplicate data entry, ensures that nerve function information is available to all healthcare providers involved in the patient's care, and facilitates longitudinal tracking across multiple clinical encounters.

The integration with EHR systems may support various workflows, including order entry for nerve function assessments, documentation of procedure details, storage of raw measurement data and derived parameters, visualization of trends over time, and incorporation of nerve function data into clinical notes and reports. By aligning with established EHR workflows, the system 10 minimizes the additional effort required for healthcare providers to incorporate nerve function assessment into their practice, potentially increasing adoption and utilization.

In some embodiments, the processor 50 may be configured to generate clinical reports that may include quantitative and/or graphical representations of nerve function parameters. These reports may present the current patient's data points alongside shaded areas representing the normal range derived from historical data. Color-coding may be used to highlight parameters that fall outside the normal range, with the degree of deviation being indicated by color intensity. This graphical approach may allow surgeons to quickly identify which nerves are potentially impaired and the specific functional aspects affected, facilitating more informed surgical planning.

The report generation capabilities of the processor 50 may extend beyond basic data presentation to include contextual interpretation and recommendations. For example, reports may include analyses of trends over time, comparisons with previous assessments, correlations with patient-reported symptoms, and suggestions for further evaluation or treatment based on the observed nerve function parameters. These interpretative elements transform raw data into actionable information, potentially enhancing the clinical utility of the nerve function assessment.

The system 10 supports comprehensive clinical assessment documentation through standardized templates and structured data collection. This standardization ensures that all relevant information is captured consistently across patients and procedures, facilitating both individual patient care and aggregate data analysis. The documentation may include procedural details (e.g., stimulation parameters, catheter positioning technique), nerve function measurements (e.g., raw data, derived parameters, normalization methods), patient responses (e.g., symptom provocation during stimulation, immediate relief following intervention), and healthcare provider observations or interpretations.

To support interdisciplinary communication, the system 10 may implement tools for sharing nerve function data and analyses among different specialists involved in a patient's care. These tools may include customizable views of the data tailored to the information needs of different specialties, secure messaging functions for discussing findings or recommendations, and collaborative annotation capabilities for highlighting specific features or patterns in the data. By facilitating this interdisciplinary communication, the system 10 supports a more coordinated approach to patient care, potentially reducing fragmentation and improving overall outcomes.

The processor 50 may incorporate a comprehensive EHR integration framework designed to facilitate bidirectional data exchange while ensuring compliance with healthcare interoperability standards. This framework may support integration with major EHR systems through standard healthcare interoperability protocols such as Health Level Seven (HL7) version 2.x messaging, HL7 Fast Healthcare Interoperability Resources (FHIR), and Digital Imaging and Communications in Medicine (DICOM) for imaging data. The use of these established standards ensures compatibility across different healthcare environments and reduces implementation complexity.

In one embodiment, the system 10 may implement an integration architecture based on the HL7 FHIR standard, which provides a modern, web-based approach to healthcare interoperability. The processor 50 may expose FHIR-compliant APIs that enable external systems to query for nerve function assessment data, while also consuming FHIR resources from EHR systems to access relevant patient information. This approach may leverage standard FHIR resources such as Patient, Observation, Procedure, and Diagnostic Report, potentially extended with custom resources or extensions to accommodate nerve function-specific data elements that are not covered by the base FHIR specification.

The data mapping and transformation framework may address the semantic and structural differences between the nerve function assessment system's internal data model and the standardized formats required for EHR integration. This framework, executed by the processor 50, may include configurable mapping templates that define how internal data elements correspond to standard codes and structures, supporting terminologies such as SNOMED CT, LOINC, and ICD-10 for clinical concepts, measurements, and diagnoses respectively. The transformation process may include data validation to ensure that outgoing data meets the requirements of the target system, error handling to address mapping exceptions, and logging to provide an audit trail of all data transformations.

The EHR integration framework may support various clinical workflows, including order entry for nerve function assessments, documentation of procedure details, and incorporation of assessment results into the patient's medical record. For order entry, the processor 50 may receive and process incoming orders from the EHR, potentially including relevant clinical information such as the reason for the assessment, the specific nerve roots to be evaluated, and any patient-specific considerations. For documentation, the processor 50 may generate structured procedure notes that capture key details of the assessment, such as the stimulation parameters used, the nerve roots assessed, and the resulting measurements. These notes may be transmitted to the EHR as discrete data elements where supported, or as formatted text for inclusion in the patient's chart.

The integration framework may address regulatory compliance requirements related to medical device connectivity and protected health information. This may include implementing appropriate authentication and authorization mechanisms to ensure that only authorized users and systems can access patient data, maintaining a comprehensive audit trail of all data access and transmission events, and ensuring that patient identifiers are handled in accordance with relevant privacy regulations. The framework may also support configurable consent models, allowing healthcare organizations to implement their specific policies regarding the sharing of nerve function assessment data with external systems.

The potential for improving outcomes and cost-effectiveness through the use of the nerve function assessment system 10 is significant. By enabling more targeted and efficient interventions, the system 10 may reduce the need for unnecessary treatments, minimize complications, and potentially shorten the overall duration of care. These improvements could lead to substantial cost savings for healthcare systems while simultaneously enhancing patient outcomes and quality of life.

For ease of description and without limitation, the terms “surgical applications” and “clinical applications” are used to distinguish contexts of use. As used herein, surgical applications refer to use during operative procedures in an operating room or analogous sterile environment (e.g., spinal decompression procedures, with or without fusion, and other percutaneous or open interventions). Clinical applications refer to use outside the operative field, including pre-surgical pain-management and diagnostic workflows (e.g., therapeutic or diagnostic injections, selective nerve root blocks, evaluations), as well as post-operative follow-up and other non-surgical interventions. These categories are not mutually exclusive, and embodiments may be suitable for one or both contexts; the terminology is not intended to limit the scope of the claims.

The term “about,” as used herein, is intended to allow for a degree of imprecision in a stated value or range, reflecting the reasonable variation that would be understood by a person of ordinary skill in the art. This variation can arise from, for example, manufacturing tolerances, measurement uncertainty, biological variability, and minor deviations that do not fundamentally change the nature or function of the subject matter being described. Where the term “about” is used in conjunction with a numerical value, it is intended to include a range of values that are insignificantly different from the stated value and would still achieve the same result or function. For instance, a current of “about 2 mA” would be understood to encompass values that are functionally equivalent to 2 mA for the purpose of eliciting a nerve response, as would be appreciated by a practitioner in the field.

The present disclosure describes a variety of features, embodiments, and aspects of the invention. It is to be understood that any of the features, embodiments, or aspects described herein can be combined with any other feature, embodiment, or aspect, unless such a combination is explicitly disclaimed or is technically infeasible. The disclosure of a particular feature in the context of one embodiment does not preclude its use in other embodiments. All possible combinations of the disclosed features are considered to be part of this disclosure, even if not explicitly recited. The invention is not limited to the specific combinations of features presented in the exemplary embodiments, and the description of such embodiments is not intended to be limiting. Any element of any embodiment is combinable with any other element of any other embodiment without departing from the scope of the invention.

As used in the claims and specification, the term “comprising” is an open-ended transitional phrase. A claim that uses “comprising” is not limited to the elements recited in the claim but may include additional, unrecited elements. The term “comprising” is inclusive and does not exclude additional, unrecited elements or method steps. The terms “including,” “having,” and “containing” are to be interpreted as being synonymous with “comprising.” In contrast, the transitional phrase “consisting of” excludes any element, step, or ingredient not specified in the claim. The transitional phrase “consisting essentially of” limits the scope of a claim to the specified materials or steps and those that do not materially affect the basic and novel characteristic(s) of the claimed invention.

The headings used in this specification are for organizational purposes only and are not to be used to interpret the meaning of the claims or specification. The detailed description is provided to enable any person skilled in the art to make and use the invention and sets forth the best modes contemplated by the inventor. The terminology used herein is for the purpose of describing particular embodiments only and is not intended to be limiting.

Unless otherwise defined, all technical and scientific terms used herein have the same meaning as commonly understood by one of ordinary skill in the art to which this invention belongs. The singular forms “a,” “an,” and “the” include plural referents unless the context clearly dictates otherwise.

The examples and embodiments described herein are for illustrative purposes only and various modifications or changes in light thereof will be suggested to persons skilled in the art and are to be included within the spirit and purview of this application and scope of the appended claims. All publications, patents, and patent applications cited herein are hereby incorporated by reference in their entirety for all purposes. In the event of a conflict between the present disclosure and any document incorporated by reference, the present disclosure controls.

It is to be understood that the invention is not limited in its application to the details of construction and to the arrangements of the components set forth in the description or illustrated in the drawings. The invention is capable of other embodiments and of being practiced and carried out in various ways. Also, it is to be understood that the phraseology and terminology employed herein are for the purpose of description and should not be regarded as limiting.

The following examples provide additional and/or alternative embodiments of the presently disclosed technology. These examples are included to provide a comprehensive and explicit basis for the subject matter of the present disclosure and are not intended to be limiting.

### Exemplary Embodiments for Longitudinal Monitoring

Example 1: A method for longitudinal assessment of nerve health performed by a system comprising a processor, the method comprising: (a) at a first time associated with a first clinical visit, determining, by the processor, a first value of a nerve-function parameter for a target nerve of a patient by (i) controlling a stimulator to apply one or more electrical stimuli proximate to the target nerve, and (ii) analyzing signals from a sensor configured to detect muscle responses evoked by the one or more electrical stimuli; (b) storing, by the processor in a database, the first value in association with a patient identifier and an indication of the first clinical visit; (c) at least one subsequent time associated with at least a second and a third clinical visit, determining, by the processor, respective second and third values of the nerve-function parameter; and (d) computing, by the processor, a longitudinal trend using the first, second, and third values to assess a change in nerve health over time.

Example 2: The method of Example 1, wherein the nerve-function parameter comprises at least one of: a stimulation threshold, a saturation threshold, a maximal response magnitude, a recruitment gain near threshold, or an electromechanical delay.

Example 3: The method of any of Examples 1-2, further comprising normalizing session values to a reference pulse width and reference frequency, and computing a within-subject z-normalized value relative to a baseline or a contralateral comparator.

Example 4: The method of any of Examples 1-3, further comprising controlling a display device to present a graphical representation of the longitudinal trend with confidence intervals, the graphical representation plotting session values on a time axis and indicating a target range for the target nerve.

Example 5: The method of any of Examples 1-4, wherein computing the longitudinal trend comprises calculating at least one of: (a) a robust slope; (b) a change-point indicator; or (c) a threshold-exceedance duration, and generating an alert when a stored criterion is met.

Example 6: The method of any of Examples 1-5, further comprising computing a composite nerve-function index as a weighted combination of two or more nerve-function parameters and trending the composite index over time.

Example 7: A system for longitudinal assessment of nerve health, the system comprising: a stimulator; a sensor configured to detect a mechanical muscle response; a database; a display; and a processor configured to determine session values of at least one nerve-function parameter, store the session values with session metadata including stimulation settings, retrieve the session values across a plurality of visits, compute a longitudinal trend, and present the trend with a status indication relative to a target range.

Example 8: The system of Example 7, wherein the sensor comprises a mechanomyography (MMG) sensor including an accelerometer, and wherein the processor validates a response only when occurring within a stimulus-locked latency window.

Example 9: The system of any of Examples 7-8, wherein the processor determines the saturation threshold by stimulating at currents above the stimulation threshold, quantifying a response magnitude, and identifying a plateau based on a slope criterion or by fitting a logistic curve.

Example 10: The method or system of any of Examples 1-9, further comprising storing, in association with each session, at least one of: stimulation parameters, sensor placement, signal-quality indices, and audit data for reproducibility of the longitudinal trend.

### Exemplary Embodiments for Impedance-Based Tissue Identification

Example 11: A method for identifying tissue during a clinical procedure, the method comprising: (a) positioning an electrode of a stimulator proximate to a target anatomical location; (b) applying, by a processor via the electrode, an alternating-current signal at one or more frequencies; (c) measuring, by the processor, a multifrequency impedance signature based on a voltage response; (d) analyzing the impedance signature to classify the tissue as one of a plurality of types including at least neural tissue and non-neural tissue; and (e) generating an alert indicative of the classified tissue type.

Example 12: The method of Example 11, wherein the plurality of tissue types further includes adipose, fibrotic/scar, muscle, and cerebrospinal fluid.

Example 13: The method of any of Examples 11-12, wherein analyzing the impedance signature comprises comparing the impedance signature to a reference database of tissue impedance characteristics and/or executing a trained classifier.

Example 14: The method of any of Examples 11-13, wherein the impedance signature includes at least one of: impedance magnitude, phase angle, or complex impedance parameters at two or more frequencies.

Example 15: The method of any of Examples 11-14, further comprising, subsequent to classifying tissue as neural tissue, determining at least one nerve-function parameter by delivering one or more electrical stimuli and detecting a corresponding muscle response.

Example 16: A system for identifying tissue during a clinical procedure, comprising: a stimulator comprising an electrode; impedance measurement circuitry; a display device; and a processor configured to apply an alternating-current signal, measure an impedance signature, classify tissue among a plurality of tissue types, present a color-coded or textual indicator, and record the classification with location metadata.

Example 17: The system of Example 16, wherein the processor analyzes the impedance signature using a machine-learning classification algorithm trained on a labeled dataset.

Example 18: The system of any of Examples 16-17, wherein the processor is further configured to gate stimulation or adjust stimulation parameters based on the classified tissue to improve safety or specificity.

Example 19: The system of any of Examples 16-18, wherein the alert comprises a visual, audible, or haptic indicator.

Example 20: The system of any of Examples 16-19, wherein the processor is further configured, subsequent to classifying neural tissue, to deliver an electrical stimulus to determine at least one nerve-function parameter.

### Exemplary Embodiments for Position Normalization via Fluoroscopy

Example 21: A method for normalizing a nerve-function assessment, comprising: (a) receiving a fluoroscopic image depicting an anatomical region including one or more anatomical landmarks; (b) positioning an electrode of a stimulator within the region; (c) identifying a location of the electrode and a location of the one or more landmarks in the image; (d) estimating an electrode-to-nerve distance based on the identified locations and an anatomical model; (e) determining a raw value of a nerve-function parameter based on a muscle response; and (f) calculating a normalized value by applying a correction factor that is a function of the estimated distance.

Example 22: The method of Example 21, further comprising displaying the normalized value with an associated distance uncertainty.

Example 23: The method of any of Examples 21-22, wherein the nerve-function parameter is a stimulation threshold or a saturation threshold, and wherein the correction factor is based on a stored distance-to-current relationship.

Example 24: The method of any of Examples 21-23, wherein the stored relationship corresponds to an approximately 1 mA per 1 mm change within a defined distance range.

Example 25: The method of any of Examples 21-24, wherein the landmarks include a pedicle, facet joint, foramen, or vertebral endplate, and wherein calibration uses at least one of DICOM pixel spacing or a radiopaque marker of known size.

Example 26: A system for normalizing a nerve-function assessment, comprising: an imaging interface; a stimulator; a sensor; and a processor configured to receive a fluoroscopic image, identify an electrode and anatomical landmarks, estimate a distance using the image and an anatomical model, determine a raw value of a nerve-function parameter, and calculate a normalized value by applying a correction factor, wherein the processor stores the normalized value together with the estimated distance and uncertainty.

Example 27: The system of Example 26, wherein the processor is configured to perform two-view triangulation using two fluoroscopic projections to reduce distance uncertainty.

Example 28: The system of any of Examples 26-27, wherein the processor identifies landmarks using a machine-learning object detector.

Example 29: The system of any of Examples 26-28, wherein the anatomical model is retrieved from a database and parameterized by spinal level.

Example 30: The system of any of Examples 26-29, wherein the nerve-function parameter is a stimulation threshold determined using a mechanical sensor.

### Exemplary Embodiments for Clinical Stimulator Designs

Example 31: A method for assessing nerve health, comprising: (a) advancing a flexible elongate probe having a plurality of spaced-apart electrodes along a distal portion; (b) selecting, by a processor, at least one electrode; (c) delivering one or more electrical stimuli proximate a target nerve; and (d) determining a value of at least one nerve-function parameter based on a detected muscle response.

Example 32: The method of Example 31, further comprising: sequentially activating at least two different electrodes; determining a respective value of the nerve-function parameter for each; and identifying an optimal value among the respective values.

Example 33: The method of any of Examples 31-32, wherein the nerve-function parameter is a stimulation threshold and the optimal value is the lowest stimulation threshold.

Example 34: The method of any of Examples 31-33, wherein the probe is advanced through a cannulated introducer needle.

Example 35: The method of any of Examples 31-34, wherein the probe is a stimulated catheter including a central lumen and further comprising delivering a therapeutic agent through the lumen subsequent to determining the nerve-function parameter.

Example 36: A system for assessing nerve health, comprising: a flexible elongate probe with a plurality of spaced-apart electrodes; a sensor configured to detect a muscle response; and a processor configured to select one or more electrodes, deliver stimuli in monopolar or bipolar configurations, and determine at least one nerve-function parameter.

Example 37: The system of Example 36, wherein the processor is configured to perform an impedance screen to exclude out-of-range contacts, execute a ladder scan across remaining contacts, and optionally perform current steering between adjacent contacts.

Example 38: The system of any of Examples 36-37, wherein the processor identifies the lowest stimulation threshold as the optimal value.

Example 39: The system of any of Examples 36-38, further comprising a display device, wherein the processor controls the display to present a spatial map of the respective values correlated to electrode positions.

Example 40: The system of any of Examples 36-39, wherein the probe is a stimulated catheter further comprising a lumen for delivering a therapeutic agent.

### Exemplary Embodiments for Predictive Analytics and Surgical Guidance

Example 41: A method for generating a clinical guidance recommendation, comprising: (a) accessing a database storing a predictive model and longitudinal nerve-function data for a patient; (b) determining a temporal trend; (c) providing the trend and patient characteristics as inputs to the predictive model; (d) generating a predicted outcome for a surgical or non-surgical pathway; and (e) generating a clinical recommendation based on the predicted outcome and the trend meeting a predefined criterion.

Example 42: The method of Example 41, wherein the predictive model is a machine-learning algorithm trained on historical longitudinal data and outcomes.

Example 43: The method of any of Examples 41-42, wherein the predefined criterion includes a rate of deterioration exceeding a threshold.

Example 44: The method of any of Examples 41-43, further comprising controlling a display device to present the recommendation with an associated confidence level and key contributing variables.

Example 45: The method of any of Examples 41-44, wherein the recommendation includes a suggested timeline for intervention.

Example 46: A system for generating a clinical guidance recommendation, comprising: a database configured to store a predictive model and longitudinal nerve-function data; and a processor configured to compute a trend, provide inputs to the model, generate a predicted outcome, generate a recommendation, and record model identifiers used to generate the recommendation.

Example 47: The system of Example 46, wherein the predictive model comprises a gradient-boosted decision-tree, random forest, or neural network.

Example 48: The system of any of Examples 46-47, further comprising a display device, wherein the processor is configured to present predicted outcomes for surgical and non-surgical options side-by-side.

Example 49: The system of any of Examples 46-48, wherein the temporal trend is a slope of a regression or robust slope fitted to values over time.

Example 50: The system of any of Examples 46-49, wherein the processor is further configured to update or re-train the predictive model with actual outcome data.

### Exemplary Embodiments for Wearable Device Integration

Example 51: A method for functional assessment of a patient, comprising: (a) determining, during a clinical visit, a value of at least one nerve-function parameter for a target nerve; (b) receiving, via a communication interface from a remote personal device, activity data collected by a wearable sensor over a period of time; (c) analyzing a correlation between the nerve-function parameter and the activity data to assess functional status; and (d) presenting an indication of the correlation.

Example 52: The method of Example 51, wherein the activity data includes at least one of: daily step count, cadence, gait asymmetry indices, sit-to-stand counts, stair counts, active minutes, or sleep metrics.

Example 53: The method of any of Examples 51-52, wherein analyzing the correlation comprises comparing a pre-visit activity summary to a post-intervention activity summary within defined time windows.

Example 54: The method of any of Examples 51-53, wherein presenting comprises a dashboard showing synchronized timelines and a radicular function index combining nerve-function parameters and activity features.

Example 55: The method of any of Examples 51-54, wherein the assessment is used to evaluate efficacy of a therapeutic intervention performed at the clinical visit.

Example 56: A system for functional assessment of a patient, comprising: a sensor configured to detect a muscle response; a communication interface configured to receive wearable activity data; a display device; and a processor configured to determine a nerve-function parameter, receive the activity data, compute at least one of a correlation coefficient or trend consistency metric, and present an indication of functional status.

Example 57: The system of Example 56, wherein the remote personal device is a smartphone and the communication interface is a wireless interface.

Example 58: The system of any of Examples 56-57, wherein the processor analyzes the correlation by executing a statistical analysis algorithm and stores a data-quality score for the wearable data.

Example 59: The system of any of Examples 56-58, wherein the processor further receives patient-reported outcome data and analyzes a relationship among the nerve-function parameter, the activity data, and the patient-reported outcome data.

Example 60: The system of any of Examples 56-59, wherein the nerve-function parameter is a stimulation threshold determined using a mechanomyography sensor.

1. A system for longitudinal assessment of nerve health comprising:
a stimulator configured to deliver electrical stimuli to tissue proximate a target nerve of a subject;
a sensor configured to detect a muscle response evoked by the electrical stimuli;
a display; and
a processor in communication with the stimulator, the sensor, and the display, the processor configured to:
control the stimulator to deliver the electrical stimuli,
determine, from the detected muscle response, one or more nerve-function parameters for the target nerve including at least a stimulation threshold,
store the one or more nerve-function parameters in association with a session identifier,
retrieve stored parameters from a plurality of sessions for the target nerve, compute a longitudinal trend across the plurality of sessions, and
control the display to present the longitudinal trend and an indication of the target nerve's status relative to a target range.

2. The system of claim 1, wherein the sensor comprises a mechanomyography sensor including an accelerometer configured to detect a mechanical muscle response.

3. The system of claim 1, wherein the sensor is supported by a carrier selected from an adhesive pad, pocketed patch, cuff, or sleeve to maintain mechanical coupling to the skin without restricting muscle motion.

4. The system of claim 1, wherein the processor is further configured to determine at least one additional nerve-function parameter selected from: a saturation threshold and a maximal response magnitude.

5. The system of claim 1, wherein the longitudinal trend comprises at least one of: a rate of change of the stimulation threshold, a change-point detection, or an alert condition triggered when the trend crosses the target range.

6. The system of claim 1, wherein the target range is individualized based on a database of historical patient data and subject-specific attributes.

7. The system of claim 6, wherein the processor executes a machine-learning model trained on the historical data to generate the individualized target range and a complication risk profile.

8. The system of claim 1, wherein the stimulator comprises a flexible multi-electrode probe, and the processor is configured to sequentially activate different electrodes to identify an electrode position that yields a lowest stimulation threshold indicative of closest proximity to the target nerve.

9. The system of claim 1, wherein the stimulator comprises a stimulated catheter having a distal electrode and a lumen for delivery of a therapeutic agent.

10. The system of claim 1, wherein the display concurrently presents the longitudinal trend and at least one of: patient-reported outcomes or activity metrics obtained from a wearable device.

11. The system of claim 1, wherein the processor is further configured to normalize a measured stimulation threshold using a fluoroscopic landmark registration that estimates an electrode-to-nerve distance and applies a correction factor to the measured stimulation threshold.

12. A method for longitudinal assessment of nerve health, comprising:
delivering electrical stimuli via a stimulator to tissue proximate a target nerve of a subject;
detecting, with a sensor, a muscle response evoked by the stimuli;
determining, by a processor, from the detected muscle response at least a stimulation threshold for the target nerve;
storing the stimulation threshold in association with a session identifier;
retrieving stimulation thresholds from a plurality of sessions for the target nerve;
computing a longitudinal trend of the stimulation thresholds across the plurality of sessions; and
presenting the longitudinal trend on a display together with an indication of the target nerve's status relative to a target range.

13. The method of claim 12, further comprising determining at least one of: a saturation threshold using an adaptive search constrained by responses above the stimulation threshold, or a maximal response magnitude at or above the saturation threshold.

14. The method of claim 12, further comprising computing a composite nerve-function index as a weighted combination of two or more nerve-function parameters to summarize the longitudinal trend.

15. The method of claim 12, further comprising receiving activity data from a wearable device platform and correlating changes in the longitudinal trend with changes in the activity data.

16. The method of claim 12, further comprising normalizing a measured stimulation threshold using fluoroscopic landmark registration that identifies anatomical landmarks and the stimulator position in an image and computes an estimated electrode-to-nerve distance applied to correct the measured stimulation threshold.

17. The method of claim 12, further comprising performing a position optimization procedure in which the stimulator is systematically repositioned while monitoring stimulation thresholds to identify a minimum stimulation threshold used as a normalized value for longitudinal comparison.

18. The method of claim 12, further comprising estimating tissue type proximate the stimulator using impedance-based differentiation and adjusting the stimulation threshold interpretation based on the estimated tissue.

19. A computer-implemented method executed by one or more servers comprising:
receiving, over a network, session records from a plurality of client systems, each session record including (i) a nerve-function parameter for a target nerve of a subject, the nerve-function parameter comprising at least a stimulation threshold determined from a sensor-detected muscle response to an electrical stimulus, and (ii) session metadata;
storing the session records in a database;
for the subject, selecting from the database a cohort of historical patients based on similarity of clinical attributes;
computing, using the database, (i) one or more individualized target ranges for the subject based on nerve-function parameters associated with successful outcomes within the selected cohort and (ii) at least one predicted outcome or complication risk for the subject; and
transmitting, to a requesting client system, data specifying the individualized target ranges, the at least one predicted outcome or complication risk, and a representation of a longitudinal trend for the subject computed from the session records for presentation by the requesting client system.

20. The method of claim 19, wherein at least one predicted outcome or risk is generated by an interpretable machine-learning model and includes an explanation identifying variables with highest contribution to the prediction.
